(19) United States
(12) Patent Application Publication
(10) Pub. No.: US 2022/0025061 A1
(43) Pub. Date: Jan. 27, 2022

(54) COMBINATION THERAPIES FOR TREATING DISEASE USING AN INNATE IMMUNITY MODIFIER AND AN OX40 AGONIST

(71) Applicant: BioXcel Therapeutics, Inc., New Haven, CT (US)

(72) Inventors: Vimal D. MEHTA, New Haven, CT (US); Luca RASTELLI, New Haven, CT (US); John MACDOUGALL, New Haven, CT (US); Snigdha GUPTA, Gurgaon (IN)

(21) Appl. No.: 17/311,770

(22) PCT Filed: Dec. 10, 2019
(86) PCT No.: PCT/US2019/065432

§ 371 Date: Jun. 8, 2021

Related U.S. Application Data
(60) Provisional application No. 62/790,569, filed on Jan. 10, 2019.
(60) Provisional application No. 62/777,350, filed on Dec. 10, 2018.

(51) Int. Cl. C07K 16/28; A61K 45/06; A61P 35/04; C07K 16/30

(52) U.S. Cl. CPC C07K 16/2878; C07K 16/2818; A61K 2039/505; A61P 35/04; C07K 16/30; A61K 45/06

(57) ABSTRACT

The present disclosure provides methods of treating cancer comprising administering a selective dipeptidyl peptidase inhibitor and an OX40 agonist with or without one or more immune checkpoint inhibitors to a subject with cancer. The present disclosure provides pharmaceutical compositions for treating cancer comprising a selective dipeptidyl peptidase inhibitor and an OX40 agonist with or without one or more immune checkpoint inhibitors.

### PRIORITY CLAIM

This application claims priority to U.S. Provisional Application Ser. No. 62/777,350, which was filed on Dec. 10, 2018 and U.S. Provisional Application Ser. No. 62/790,569, which was filed on Jan. 10, 2019, the disclosures of which are hereby incorporated by reference in their entirety.

### TECHNICAL FIELD

The present disclosure relates to combination therapies for the treatment of cancer. In particular, the present disclosure relates to methods of treating cancer by administering to a subject a selective dipeptidyl peptidase inhibitor and an OX40 agonist with or without an immune checkpoint inhibitor. The present disclosure also provides pharmaceutical compositions comprising a selective dipeptidyl peptidase inhibitor and an OX40 agonist with or without an immune checkpoint inhibitor.

### BACKGROUND

Tumor cells have the capacity to rapidly grow and metastasize by suppressing, evading, and exploiting the host immune system. Immunotherapy is a form of oncologic treatment directed towards enhancing the host immune system against cancer. In recent years, suppression of immune checkpoints, such as cytotoxic T lymphocyte-associated molecule-4 (CTLA-4), programmed cell death receptor-1 (PD-1), and programmed cell death ligand-1 (PD-L1) has emerged as an important and effective form of immunotherapy (Marin-Acevedo J A et al, Cancer immunotherapy beyond immune checkpoint inhibitors, J. Hematol Oncol. 2018 Jan. 12; 11(1):8). These immune checkpoint inhibitors have produced impressive results in the clinic in a wide range of cancers, leading to FDA approval for various types of cancer. Although ICIs have improved cancer outcomes, they are not an effective treatment option for all types of cancer. In addition, some patients initially respond to immune checkpoint therapy, but then relapse due to the emergence of resistant pathways. Further, immune checkpoint therapy can have adverse side effects and even result in death (Gajewski T F, Semin Oncol. 2015 August; 42(4):663-71; Gide T N et. al., Clin Cancer Res. 2018 Mar. 15; 24(6):1260-1270; Moslehi J J et al., Lancet. 2018 Mar. 10; 391(10124):93; Heinzerling L et al., J Immunother Cancer. 2016 Aug. 16; 4:50).

For at least these reasons, there is a continuing need to identify new approaches for the treatment of cancer.

### SUMMARY

The present disclosure provides novel cancer therapies comprising an inhibitor of dipeptidyl peptidase 4 activity and/or structure homologues (DASH) proteases and an OX40 agonist with or without an immune checkpoint inhibitor. Inhibition of DASH proteases results in pyroptosis of tumor-associated macrophages, which drives activation of caspase 1 and the release of IL-1β, and perhaps other immunostimulatory cytokines, including IL-18. This results in the redistribution and altered activity of tumor-associated MDSCs, enhanced priming of T cells and dendritic cells, and trafficking of T cells and other immune cells to the tumor microenvironment.

In the methods and compositions disclosed herein, the selective dipeptidyl peptidase inhibitor (e.g., talabostat) is combined with an OX40 agonist. Without being bound by theory, it is thought that administering a selective dipeptidyl peptidase inhibitor induces immunostimulatory cytokines such as IL-18, which increases the activation of CD4⁺ helper T cells and CD8⁺ cytotoxic T cells, leading to the upregulation of OX40 ligand and OX40. Thus, when the selective dipeptidyl peptidase inhibitor is combined with an OX40 agonist, T cell activation is enhanced, resulting in synergistic anti-tumor activity, reduced tumor growth and increased survival. The present disclosure also provides for a selective dipeptidyl peptidase inhibitor and OX40 agonist further combined with an immune checkpoint inhibitor. The triple combination provides a particularly robust response against cancer by further enhancing T cell activity and reducing immunosuppression in the tumor microenvironment. In particular, talabostat mesylate in combination with an OX40 agonist antibody with or without an anti-PD-1 antibody results in reduced tumor burden and increased survival against solid cancers (e.g., colorectal cancer).

In embodiments, the present disclosure relates to a method of treating cancer comprising administering to a subject therapeutically effective amounts of a selective dipeptidyl peptidase inhibitor (e.g., talabostat) and an OX40 agonist with or without one or more immune checkpoint inhibitors.

In embodiments, the therapeutic agents are administered to the cancer subject at the same time (separately or together as part of a single pharmaceutical formulation), sequentially in any appropriate order, or intermittently. When administered separately, each therapeutic agent is prepared as a separate pharmaceutical composition suitable for administration via the appropriate administration route.

In embodiments, the present disclosure provides pharmaceutical compositions comprising a selective dipeptidyl peptidase inhibitor (e.g., talabostat) and an OX40 agonist. In other embodiments, the pharmaceutical composition comprises a selective dipeptidyl peptidase inhibitor (e.g., talabostat), an OX40 agonist and one or more immune checkpoint inhibitors. The pharmaceutical compositions disclosed herein are formulated with one or more pharmaceutically acceptable carriers and/or excipients.

In embodiments, the present disclosure provides a kit for treating a subject with cancer, the kit comprising a selective dipeptidyl peptidase inhibitor (e.g., talabostat) and an OX40 agonist. In other embodiments, the present disclosure provides a kit for treating a subject with cancer, the kit comprising a selective dipeptidyl peptidase inhibitor (e.g., talabostat), an OX40 agonist and one or more immune checkpoint inhibitors. instructions for using said selective dipeptidyl peptidase inhibitor, OX40 agonist and/or immune checkpoint inhibitor(s) according to the methods described herein.

### BRIEF DESCRIPTION OF THE DRAWINGS

FIG. 1 shows a plot of mean tumor volume versus time in mice following treatment with various combinations of talabostat mesylate, anti-mouse PD-1 antibody, and/or anti-mouse OX40 agonist antibody in a MC38 mouse model of colon adenocarcinoma as described in Example 1. Group 1=vehicle control, Group 2=talabostat mesylate (20 μg per mouse, qd), Group 3=anti-OX40 agonist antibody (10 mg/kg; twice weekly), Group 4=talabostat mesylate (20 μg per mouse, qd) and anti-PD-1 antibody (10 mg/kg twice weekly), Group 5=anti-PD-1 antibody 5 mg/kg twice weekly) and anti-OX40 agonist antibody (10 mg/kg; twice weekly), Group 6=talabostat mesylate (20 μg per mouse, qd) and anti-OX40 antibody (10 mg/kg; twice weekly) and Group 7=talabostat mesylate (20 μg per mouse, qd), anti-OX40 antibody (10 mg/kg; twice weekly) and anti-PD-1 antibody (5 mg/kg twice weekly). Tumor size was measured up to day 23 after tumor inoculation.

FIG. 2 shows a plot of percent survival versus time in mice following treatment with various combinations of talabostat mesylate, anti-mouse PD-1 antibody, and anti-mouse OX40 agonist antibody in a MC38 mouse model of colon adenocarcinoma as described in Example 1

### DETAILED DESCRIPTION

### Abbreviations

Ab: Antibody

CTLA4: Cytotoxic T-lymphocyte-associated antigen 4

DPP8: Dipeptidyl peptidase-8

DPP9: Dipeptidyl peptidase-9

FAP: Fibroblast activation protein

ICI: Immune checkpoint inhibitor

I.V. Intravenous

P.O: Per Oral

PD-1: Programmed Cell Death 1

PD-L1: Programmed Cell Death Ligand 1

PD-L2: Programmed Cell Death Ligand 2

Q.D: Quaque die

TME: Tumor microenvironment

TNFRSF4: Tumor necrosis factor receptor superfamily, member 4

Microgram: mcg or μg

### Definitions

Various terms are used throughout the specification and claims. Such terms are to be given their ordinary meaning in the art unless otherwise indicated. Other specifically defined terms are to be construed in a manner consistent with the definition provided herein.

As used herein, the phrase “therapeutically effective amount” refers to the quantity of a component or of a combination, which is sufficient to yield a desired therapeutic response, for example, a reduction in tumor growth or in tumor size, without undue adverse side effects (such as, for example, toxicity, irritation, or allergic response) commensurate with a reasonable benefit/risk ratio when used in the manner of this disclosure. The therapeutically effective amount will vary with factors such as the particular condition being treated, the physical condition of the patient, the type of mammal or animal being treated, the duration of the treatment, the nature of concurrent therapy, and the specific formulations employed, and the types of therapeutic agents being administered.

The terms “subject” and “patient” are used interchangeably herein, and refer to any animal amenable to the methods or compositions described herein. In certain non-limiting embodiments, the subject or patient is a primate, a rodent, a cat, a dog, a rabbit, a cow, a horse, a goat, a sheep, or a pig. Exemplary rodents are mouse, rat, hamster, and guinea pig. Exemplary primates are monkey, chimpanzee, orangutan, gorilla, and human. Typically, the primate is a human.

The term “treating” within the context of the present disclosure, means an alleviation of symptoms associated with a disorder or disease, or halt of further progression or worsening of those symptoms, or prevention or prophylaxis of the disease or disorder. For example, “treating” cancer using the methods and compositions of the present disclosure may include a reduction in tumor growth, reduced metastasis and/or increased survival.

As used herein the term “cancer” can be used interchangeably with “tumor”. The term “cancer” or “tumor” refers to cancers of any type including both solid tumors and non-solid tumors, such as leukemia and lymphoma. Carcinomas, sarcomas, myelomas, lymphomas, and leukemia can all be treated using the present disclosure.

The terms “inhibitor” or “antagonist” refers to a molecule that blocks or negatively modulates the activity of another biologically active molecule. Antagonists or inhibitors include, but are not limited to, small organic molecules, ions, proteins, nucleic acids, carbohydrates, lipids, or any other molecules that bind to or interact with biologically active molecules.

The term “agonist” refers to a molecule that enhances or increases the biological activity of another molecule. Agonists may include proteins, peptides, nucleic acids, carbohydrates, small molecules (e.g., such as metabolites), aptamers or other compounds or compositions that modulate the activity of another biological molecule.

The term “agonistic antibody” as used herein refers to an antibody which, when bound to a receptor, e.g. the OX40 receptor is capable of stimulating biological activity that is similar to or the same as the receptor's natural ligand, e.g., the OX40 ligand. For example, an OX40 agonistic antibody is capable binding to the OX40 receptor on activated CD4+ T-cells and stimulating activation of the signal transduction pathway associated with the OX40 receptor.

The term “aptamer” as used herein refers to a nucleic acid or peptide that is capable of specifically binding to target molecules (through hydrogen bonding, electrostatic complementarity, hydrophobic contacts and/or steric exclusion), thereby modulating the activity of the target molecule.

The term “multimeric protein” as used herein refers to a protein composed of two or more proteins or polypeptides. A multimeric protein is meant to include any heterodimeric or hetero-oligomeric protein, e.g., a heterodimeric cell surface or nuclear receptor. Multimeric protein receptors encompass both soluble and membrane forms of the receptor.

Oligomeric proteins as defined herein refers to proteins that are composed of more than one subunit (polypeptide chain) and possess a quaternary structure. These proteins may be composed either exclusively of several copies of identical polypeptide chains, in which case they are termed homo-oligomers, or alternatively by at least one copy of a different polypeptide chain (hetero-oligomers).

The term “fusion protein” refers to a protein or polypeptide having an amino acid sequence derived from two or more proteins.

As used herein, the term “immunoadhesin” refers to an antibody-like molecule which combines the “binding domain” of a heterologous protein (an “adhesin”, e.g. a receptor, ligand or enzyme) with the effector functions of immunoglobulin constant domains. Structurally, the immunoadhesins comprise a fusion of the adhesin amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesins may be obtained from any immunoglobulin, such as IgG1, IgG2, IgG3, or IgG4 subtypes, IgA, IgE, IgD or IgM.

As used herein, the term “antibody” refers to an immunoglobulin or fragment thereof, and encompasses any polypeptide comprising an antigen-binding site regardless of the source, species of origin, method of production, and characteristics. Antibodies may be comprised of heavy and/or light chains or fragments thereof. The antibodies or antigen-binding fragments, variants, or derivatives thereof may be polyclonal, monoclonal, multi-specific, human, humanized, primatized, or chimeric antibodies, single chain antibodies, epitope-binding fragments, e.g., Fab, Fab′, F(ab′)2, Fv, Fd, single-chain Fv (scFv), disulfide-linked Fvs (sdFv), VL, VH, camel Ig, V-NAR, VHH, trispecific (Fab3), bispecific (Fab2), diabody ((VL-VH)2 or (VH-VL)2), triabody (trivalent), tetrabody (tetravalent), minibody ((scFv-CH3)2), a nanobody, bispecific single-chain Fv (Bis-scFv), IgGdeltaCH2, scFv-Fc or (scFv)2-Fc, fragments comprising either a VL or VH domain, fragments produced by a Fab expression library, and anti-idiotypic (anti-Id) antibodies. ScFv molecules are known in the art and are described in U.S. Pat. No. 5,892,019. Immunoglobulin or antibody molecules of the present disclosure can be of any type (e.g., IgG, IgE, IgM, IgD, IgA, and IgY), class (e.g., IgG1, IgG2, IgG3, IgG4, IgA1 and IgA2) or subclass of an immunoglobulin molecule. In embodiments, the antibody is a nanobody. Examples of nanobodies are described in U.S. Pat. Nos. 5,800,988 and 6,005,079 and PCT Application Publication Nos. WO1994/04678, 1994/25591 and European Application Publication No. EP2673297, each of which are incorporated herein by reference.

As used herein, the term “pharmaceutically acceptable” means approved by a government regulatory agency or listed in the U.S. Pharmacopoeia or another generally recognized pharmacopoeia for use in the subject, particularly in humans.

The term “pharmaceutically acceptable salt” refers to salts derived from a variety of organic and inorganic counter ions well known in the art. Reference to compounds herein is meant to encompass pharmaceutically acceptable salt forms, as appropriate. Pharmaceutically acceptable acid addition salts may be formed with inorganic acids and organic acids. For reviews of suitable salts, see, e.g., Berge, et al., J. Pharm. Sci. 66:1-19 (1977) and Remington: The Science and Practice of Pharmacy, 20th Ed., ed. A. Gennaro, Lippincott Williams & Wilkins, 2000. Non-limiting examples of suitable acid salts includes: hydrochloric acid, hydrobromic acid, sulfuric acid, nitric acid, phosphoric acid, acetic acid, propionic acid, glycolic acid, pyruvic acid, oxalic acid, maleic acid, malonic acid, succinic acid, lactate acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Non-limiting examples of suitable base salts includes: sodium, potassium, lithium, ammonium, calcium, magnesium, iron, zinc, copper, manganese, aluminium, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Compounds described herein, when containing one or more chiral centers, are meant to encompass all stereoisomeric forms and mixtures thereof, including enantiomers, diastereoisomers, racemic mixtures, mixtures of enantiomers where one enantiomer is present in enantiomeric excess, and the like.

As used herein, the term “combination” may refer to a composition containing at least two therapeutic agents, wherein each therapeutic agent may be referred to as a component of the combination. The term “combination” may also refer to a method or administration regime in which multiple therapeutic agents are administered simultaneously or sequentially in separate compositions. Such sequential administrations are separated by a period of time.

### Dipeptidyl Peptidase Inhibitors

The term “innate immunity modifier” as used herein refers to a selective dipeptidyl peptidase (DPP) inhibitor targeting DPP8, DPP9 and/or FAP. DPPs are a class of serine proteases encoded by the DPP gene (classified under EC 3.4.14). There are 9 types of DPP genes known to date, which include cathepsin C (DPP-1), DPP-2, DPP-3, DPP-4, DPP-6, DPP-7, DPP-8, DPP-9, DPP-10 and fibroblast activation protein (FAP).

In embodiments, the selective dipeptidyl peptidase inhibitor is a compound. In embodiments, the selective dipeptidyl peptidase inhibitor is talabostat. In embodiments, the selective dipeptidyl peptidase inhibitor is a pharmaceutically acceptable salt of talabostat; for example, talabostat mesylate. Talabostat mesylate has a CAS registration number of 150080-09-4. In embodiments, the selective dipeptidyl peptidase inhibitor is an analog, prodrug or stereoisomer of talabostat. Talabostat analogs include compounds such as ARI-4175 disclosed in EP Patent No. 2,782,994 and talabostat-like boro-Pro compounds disclosed in PCT Application Publication Nos. WO2018/049014 and WO2018/049008. Prodrugs of talabostat include compounds such as cyclohexyl(glycinyl)-prolinyl-valinyl-L-boroproline disclosed in PCT Application Publication No. WO2003/092605. Talabostat stereoisomers include compounds disclosed in PCT Application Publication No. WO1993/008259 and U.S. Pat. No. 6,825,169.

In embodiments, the dipeptidyl peptidase inhibitor is a compound that inhibits FAP. Examples of FAP inhibitors include, but are not limited to: ARI-3099 (N-(pyridine-4-carbonyl)-d-Ala-boroPro) as disclosed Poplawski et al., 2013, Vol. 56(9) pp. 3467-3477; ARI-3996 as disclosed in U.S. Patent Application Publication No. 20140255300; MIP-1231 (MIP-1232 or MIP-1233) as disclosed in U.S. Patent Application Publication No. 20100098633; (4-quinolinoyl)-glycyl-2-cyanopyrrolidines as disclosed by Jansen et. al., 2013, ACS Med Chem Lett, Vol. 4 (5), page no. 491-496; (2S)-1-(2-(1-Napthoylamino)acetyl)pyrrolidine-2-carbonitrile as disclosed in U.S. Pat. No. 8,183,280; (S)-A-(2-(2-cyano-4,4-difluoropyrrolidin-1-yl)-2-oxoethyl)-1-naphthamide and other related derivatives as disclosed in PCT Application Publication No. WO2013/107820; (2S)-1-((2 S)-2-(2-Methoxybenzoylamino)-3-methylpentanoyl) pyrrolidine-2-carbonitrile and other related derivatives as disclosed in U.S. Patent Application Publication No. 20120053222; Ac-Gly-BoroPro as disclosed by Edosada et al. 2006, Journal of Biological Chemistry, Vol. 281(11) page no. 7437-7444; Substituted 4-carboxylmethyl pyroglutamic acid diamides as disclosed by Tsai et al., 2010, Journal of Medicinal Chemistry, Vol. 53(18), 6572-6583; GEH200200 as disclosed by Iveson et al., 2014, Vol. 41(7), 620; UAMC-1110 as disclosed in U.S. Pat. No. 9,346,814; as well as FAP inhibitors also disclosed in PCT Application Publication No. WO2002/038590 and U.S. Pat. Nos. 7,399,869 and 7,998,997.

In embodiments, the selective dipeptidyl peptidase inhibitor is an antibody. In embodiments, the selective dipeptidyl peptidase inhibitor is an antibody that inhibits FAP. In embodiments, the FAP antibody is sibrotuzumab. Other examples of FAP antibodies are described in U.S. Pat. No. 8,568,727, European Patent No. 1,268,550, U.S. Pat. Nos. 8,999,342 and 9,011,847. Additional FAP inhibitors include bispecific antibodies of FAP (e.g., FAP-DR-5 antibody) such as disclosed in U.S. Patent Appl. Publication Nos. 2014/0370019 and 2012/0184718. Also suitable for use in the present disclosure is a chimeric antigen receptor which comprises an anti-FAP domain such as disclosed in U.S. Patent Appl. Publication No. 20140099340.

### OX40 Agonists

OX40 (also known as CD134, TNFRSF4, ACT4, ACT35, IMD16 and TXGP1L), is a member of the tumor necrosis factor receptor superfamily. The term “OX40” as used herein includes any variants or isoforms of OX40 which are naturally expressed by cells. OX40 is induced on T cells after engagement of the T cell receptor. The ligand for OX40 (OX40L) is predominantly expressed on antigen presenting cells. OX40 is highly expressed by activated CD4+ T cells, activated CD8+ T cells, memory T cells, and regulatory T (Treg) cells. OX40-OX40L signalling in activated CD4+ and CD8+ T cells leads to enhanced proliferation, survival, effector function, memory development and migration.

OX40 agonists useful in the methods and compositions of the present disclosure are selected from the group consisting of an antibody, a fusion protein, an oligomeric or multimeric molecule, an aptamer, an OX40L agonist fragment, and an immunoadhesin, preferably an antibody.

In embodiments, the OX40 agonist is an antibody. Exemplary OX40 antibodies include, without limitation, human OX40 antibodies, mammalian OX40 antibodies, humanized OX40 antibodies, fully humanized OX40 antibodies, monoclonal OX40 antibodies, polyclonal OX40 antibodies, chimeric OX40 antibodies, OX40 domain antibodies, single chain OX40 antibody fragments, heavy chain OX40 antibody fragments or light chain OX40 antibody fragments.

In embodiments, the OX40 antibody is selected from the group consisting of PF-04518600, pogalizumab (vonlerolizumab, MOXR0916, or RG-7888), MEDI6469, OX86, L106, ACT35, MEDI0562 (tavolixizumab or tavolimab), INCAGN01949, ABBV368 and GSK3174998. In embodiments, the OX40 antibody is PF-04518600.

In embodiments, the OX40 agonist is a fusion protein. In embodiments, the OX40 agonist is efizonerimod alfa (MEDI 6383). In other embodiments, the OX40 fusion protein is PD1-Fc-OX40L.

In embodiments, the OX40 agonist is an aptamer. OX40 aptamers and examples thereof are described in WO2008/048685, incorporated herein by reference in its entirety.

In embodiments, the OX40 agonist is a single-chain fusion protein comprising three soluble OX40L domains and an Fc fragment. Single-chain OX40 agonist proteins and examples thereof are further described in WO2017/068181, incorporated herein by reference in its entirety.

In embodiments, the OX40 agonist is an immunoadhesin. In embodiments, the OX40 immunoadhesin is a trimeric OX40-Fc protein. For example, the OX40 agonist may include one or more extracellular domains of OX40L linked to an immunoglobulin Fc domain and a trimerization domain (including, without limitation, an isoleucine zipper domain). OX40 immunoadhesins are further described in US2015/0190506 and U.S. Pat. No. 7,959,925, incorporated herein by reference in their entirety.

In embodiments, the OX40 agonist is a multimeric binding molecule. In embodiments, the multimeric binding molecule includes at least three, at least four, at least five, at least six, at least seven, at least eight, at least nine, at least ten, at least eleven, or twelve antigen-binding domains that specifically and agonistically bind to an OX40 monomer expressed on the surface of the cell, thereby activating OX40-mediated signal transduction in the cell. In certain aspects, the three, four, five, six, seven, eight, nine, ten, eleven, or twelve antigen-binding domains bind to the same extracellular OX40 epitope. In certain aspects, the three, four, five, six, seven, eight, nine, ten, eleven, or twelve antigen-binding domains each specifically bind one of a group of two or more different extracellular OX40 epitopes. OX40 multimers and examples thereof are further described in WO2018/017888, incorporated herein by reference in its entirety.

Other examples of OX40 agonist antibodies useful for the present disclosure are described in U.S. Pat. Nos. 7,960,515; 10,150,815; 9,738,723; 7,550,140; and 7,696,175; U.S. Patent Application Publication No. 2015/0190506; PCT Patent Application Publication Nos. WO2009/079335; WO2013/02823; WO2013/119202; WO2012/027328; WO2013/028231; WO2013/038191; and, WO2014/148895; and European Patent No. EP0672141 B1, each of which is incorporated by reference herein in its entirety.

Accordingly, the OX40 antibodies described herein may cross-react with OX40 from species other than human (e.g., cynomolgus OX40). Alternatively, the antibodies may be specific for human OX40 and may not exhibit any cross-reactivity with other species.

In embodiments, the anti-human OX40 agonist antibody has a functional Fc region. In embodiments, the Fc region is human IgG1 or IgG4. In embodiments, the anti-human OX40 agonist antibody is engineered to increase effector function (e.g., compared to the effector function in a wild-type IgG1). In embodiments, the antibody has increased binding to a Fcγ receptor. In embodiments, the antibody lacks fucose attached to the Fc region (either directly or indirectly).

In embodiments, an OX40 agonist for use in the present disclosure is a fusion protein. In embodiments, an OX40 agonist may be a trimeric OX40L fusion protein. For example, an OX40 agonist may include one or more extracellular domains of OX40L linked to an immunoglobulin Fc domain and a trimerization domain (including without limitation an isoleucine zipper domain). In embodiments, an OX40 agonist may be linked to another protein domain, which enhances its stimulatory activity, half-life, or other desired characteristics. In embodiments, an OX40 agonist may include one or more extracellular domains of OX40L linked to an immunoglobulin Fc domain.

In embodiments, the OX40 agonist may include one or more extracellular domains of OX40L. Examples of extracellular domains of OX40L may include OX40-binding domains. In embodiments, an OX40 agonist may be a soluble form of OX40L that includes one or more extracellular domains of OX40L but lacks other, insoluble domains of the protein (e.g., transmembrane domains).

### Immune Checkpoint Inhibitors

As described herein, an “immune checkpoint” refers to an immune pathway that under normal physiological conditions regulates uncontrolled immune reactions and thus plays a role in the maintenance of self-tolerance and tissue protection. Examples of immune checkpoints molecules that regulate immune checkpoint pathways include, but are not limited to, CTLA-4, PD-1, PDL-1, and PDL-2

An “immune checkpoint inhibitor” refers to a molecule that reduces or eliminates the activity of an immune checkpoint molecule as defined above.

In embodiments, the immune checkpoint inhibitor is an antibody. In embodiments, the immune checkpoint inhibitor is an antibody with three or more complementary determining regions (CDRs). In embodiments, the antibody is directed against PD-1, PD-L1, PD-L2 or CTLA4. For example, in some embodiments, the anti-PD-1, PD-L1, PD-L2 or CTLA-4 antibody may be TECENTRIQ® (atezolizumab), KEYTRUDA® (pembrolizumab), BAVENCIO® (avelumab), IMFINZI® (durvalumab), OPDIVO® (nivolumab) and/or YERVOY® (ipilimumab).

### PD-1 Axis Inhibitors:

As described herein, the term “PD-1 axis” refers to molecules involved in the PD-1 signalling pathway, including, but not limited to, PD-1, PD-L1 and PD-L1.

Programmed death 1 (PD-1, also known as CD279 and PDCD1) is an inhibitory member of the CD28 family of receptors and as described herein, includes variants, isoforms, and species homologs of human PD-1. The complete human PD-1 sequences can be found under GenBank Accession No. U64863 and UniPro ID: Q15116.

Two ligands for PD-1 have been identified, programmed cell death ligand 1 (PD-L1, also known as PDCD1L1, PDCD1LG1, CD274 or B7H1) and programmed cell death ligand 2 (PD-L2, also known as PDCD1L2, PDCD1LG2, CD273 and B7DC), and as described herein, include variants, isoforms, species homologs, and analogs having at least one common epitope with human PD-L1 or PD-L2. PD-L1 and PD-L2 have been shown to suppress T cell activation upon binding to PD-1 (Freeman et al., (2000) J Exp. Med. 192: 1027-34; Latchman et. al. (2001) Nat Immunol. 2:261-8; Carter et al. (2002) Eur. J Immunol 32:634-43).

As described herein, a PD-1 axis inhibitor binds directly to the PD-1 receptor and prevents or blocks binding of PD-1 ligands such as PD-L1 or PD-L2. In other embodiments, the PD-1 axis inhibitor binds to PD-L1 or PD-L2 and reduces or eliminates the binding of these ligands to the PD-1 receptor, thereby preventing T cell suppression. Examples of PD-1 axis inhibitors that can be employed in the methods and compositions of the present disclosure are described in U.S. Pat. No. 8,609,089 and U.S. Patent Appl. Publication Nos. 2010/0028330 and 2012/0114649.

In embodiments, the PD-1 axis inhibitor is an anti-PD-1 antibody. Exemplary anti-PD-1 antibodies that may be used in the methods and compositions of the present disclosure include, but are not limited to, ANA011, AUNP-12, pembrolizumab, MCLA-134, mDX400, MEDI0680, muDX400, nivolumab, sasanlimab (PF-06801591), STI-A1110, dostarlimab (TSR-042 or TSR042 or ANB011), 244C8, 388D4, prolgolimab (BCD100), camrelizumab (SHR 1210), cetrelimab (JNJ63723283), JS001, spartalizumab (PDR 001), cemiplimab (semiprimab, REGN 2810), tislelizumab (BGB-A317), AMP-224, XCE853, GLS-010 (AB-122; WBP-3055), sintilimab (IBI-308), genolimzumab (CBT-501, GB226, APL-501), AK-103, theralizumab (TGN1412, CD28-SuperMAB, TAB-08 and TAB08), BI-754091, INCMGA00012 (MGA 012, INCMGA-0012), ABBV-181 (budigalimab), CC-90006 (C-90006), AGEN-2034w (AGEN-2034), LZM-009, Sym021, AK-105, CS1003, HLX-10 and AMP-224. In embodiments, the anti-PD-1 antibody is selected from pembrolizumab, nivolumab, tislelizumab (BGB-A317), MEDI0680, spartalizumab (PDR001), sasanlimab (PF-06801591), cemiplimab (REGN 2810), camrelizumab (SHR 1210), AMP-224 and dostarlimab (TSR-042). The anti-PD-1 antibodies disclosed herein may be procured from BPS Biosciences, BioXCell or other commercial sources.

In embodiments, the anti-PD-1 antibody is nivolumab (also known as OPDIVO®, MDX-1106, MDX-1106-04, ONO-4538 or BMS-936558). Nivolumab is a fully humanized IgG4 (S228P) anti-PD-1 antibody which selectively prevents interaction with PD-1 ligands (PD-L1 and PD-L2), thereby promoting anti-tumor T cell functions (U.S. Pat. No. 8,008,449; PCT Application Publication No. WO2006/121168; Wang et al., Cancer Immunol Res. 2:846-56 (2014); Topalian, S. L. et al., N Engl J Med 366.2443-2454 (2012); Topalian, S. L. et al, Current Opinion in Immunology 24:207-212 (2012); Topalian, S. L. et al, J Clin Oncol 31 (suppl):3002 (2013)). Nivolumab has been approved by the U.S. FDA for the treatment of patients with unresectable or metastatic melanoma, metastatic squamous non-small cell lung cancer, advanced renal cell carcinoma, and classical Hodgkin lymphoma.

In embodiments, the anti-PD-1 antibody is pembrolizumab (also known as KEYTRUDA®, lambrolizumab, SCH-900475 and MK-3475). Pembrolizumab is a humanized monoclonal IgG4 kappa antibody directed against PD-1. Pembrolizumab is described, for example, in U.S. Pat. Nos. 8,354,509 and 8,900,587; PCT Application Publication No. WO2009/114335 and Hamid, O. et. al, N Engl J Med 369: 134-144 (2013). Pembrolizumab has been approved by the U.S. FDA for the treatment of patients with advanced melanoma, non-small cell lung cancer, and head and neck squamous cell cancer. See, Poole, R. M., Drugs 74: 1973-1981 (2014).

In embodiments, the PD-1 inhibitor is a fusion protein. In embodiments, the PD-1 inhibitor is the PD-L2-Fc fusion protein AMP-224, which binds to and inhibits PD-1. AMP-224 is described in PCT Application Publication Nos. WO2010/027827 and WO2011/066342.

In embodiments, the PD-L1 inhibitor is an antibody. Exemplary anti-PD-L1 antibodies used in the methods and compositions of the present disclosure include, but are not limited to, avelumab (MSB0010718C), MDX-1105 (BMS-936559), CK-301 (cosibelimab), lodapolimab (LY-3300054), CX-072, CBT-502 (TQB2450), FAZ-053, FS118, HTI-1088 (HTI-131 and SHR 1316), MSB 2311, BGB-A333, IMC-001(STI-3031, STI-A1015, KN035), HLX-20, A167 (HBM-9167, KL-A167), KD033, durvalumab (MEDI4736), MCLA-145, SP142, STI-A1011, STIA1012, STI-A1010, STI-A1014, A110, KY1003 and atezolizumab (MDPL3280A, YW243.55.570). In embodiments, the anti-PD-L1 antibody is selected from the group consisting of avelumab, durvalumab and atezolizumab. In embodiments, the anti-PD-L1 antibody is avelumab.

Additional PD-L1 inhibitors are described in U.S. Pat. No. 8,217,149; PCT Application Publication Nos. WO2007/005874; WO2011/066389; WO2015/033301; WO2015/033299; and U.S. Patent Appl. Publication Nos. 2013/0034559 and 2014/0341917.

In embodiments, the PD-L2 inhibitor is an antibody. In embodiments, the anti-PD-L2 antibody is rHIgM12B7.

In embodiments, the PD-1 axis inhibitor is a compound. In embodiments, the compound inhibits PD-1, PD-L1 and/or PD-L2. In embodiments, the compound used for the methods and compositions disclosed herein is selected from the group consisting of CA-170, tomivosertib, INCB086550, BMS-103 and BMS-142. In embodiments, the compound is CA-170. CA-170 is a small molecule that selectively targets and inhibits PD-L1, PD-L2, and V-domain immunoglobulin suppressor of T-cell activation (VISTA).

### CTLA4 Inhibitors:

In embodiments, the immune checkpoint inhibitor reduces or eliminates the activity of CTLA4. In embodiments, the CTLA4 inhibitor is an antibody. In other embodiments, the CTLA4 inhibitor is a compound.

In embodiments the CTLA4 inhibitor is an antibody. Exemplary anti-CTLA4 antibodies used for the methods and compositions of the present disclosure include, but are not limited to, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, chimeric anti-CTLA4 antibodies, MDX-010 (ipilimumab), tremelimumab, anti-CTLA4 adnectins, anti-CTLA4 domain antibodies, single chain anti-CTLA4 fragments, heavy chain anti-CTLA4 fragments and light chain anti-CTLA4 fragments.

In embodiments, the anti-CTLA4 antibody is the human monoclonal antibody 10D1 (also referred to as MDX-010 or ipilimumab, available from Medarex, Inc., Bloomsbury, N.J.). Additional disclosure on 10D1 is described in PCT Application Publication No. WO2001/014424. In other embodiments, the anti-CTLA4 antibody is tremelimumab. Other CTLA4 antibodies used in the methods and compositions of the present disclosure include, but are not limited to, KAHR-102, AGEN1884, BMS-986218, MK-1308, ADU-1604, BMS-986249, CS-1002, BCD-145, REGN-4659 and KN044.

In embodiments, the CTLA4 inhibitor is a compound, i.e. small molecule. Examples of CTLA4 compound inhibitors that can be used in the methods and compositions of the present disclosure include, but are not limited to, CCC07-01, KULA101, BPI-002 and APV0437.

Additional disclosure on CTLA4 antagonists are described in PCT Application Publication Nos. WO98/42752 and WO2004/035607; U.S. Pat. Nos. 5,811,097; 5,855,887; 5,977,318; 6,051,227; 6,682,736; 6,207,156; 6,984,720; 7,109,003, and 7,132,281; U.S. Patent Appl. Publication Nos. 2005/0201994; 2002/0039581; and 2002/086014; European Patent No. 1212422B; Hurwitz et al., Proc. Natl. Acad. Sci. USA, 95(17): 10067-10071 (1998); Camacho et al., J. Clin: Oncology, 22(145): Abstract No. 2505 (2004) (antibody CP-675206); and Mokyr et al., Cancer Res., 58:5301-5304 (1998).

### Methods of Treatment

The present disclosure provides a method of treating a cancer in a subject comprising administering to the subject therapeutically effective amounts of a selective dipeptidyl peptidase inhibitor and an OX40 agonist. In embodiments, the method of treating cancer in a subject comprises administering to the subject therapeutically effective amounts of a selective dipeptidyl peptidase inhibitor, an OX40 agonist and one or more immune checkpoint inhibitors.

In embodiments, the subject with cancer is administered therapeutically effective amounts of a selective dipeptidyl peptidase inhibitor and an OX40 agonist, wherein the selective dipeptidyl peptidase inhibitor is talabostat and the OX40 agonist is PF-04518600.

In embodiments, the subject with cancer is administered therapeutically effective amounts of a selective dipeptidyl peptidase inhibitor, an OX40 agonist and an immune checkpoint inhibitor, wherein the selective dipeptidyl peptidase inhibitor is talabostat, the OX40 agonist is PF-04518600, and the immune checkpoint inhibitor is a PD-1 axis inhibitor.

In embodiments, the subject with cancer is administered therapeutically effective amounts of a selective dipeptidyl peptidase inhibitor, an OX40 agonist and an immune checkpoint inhibitor, wherein the selective dipeptidyl peptidase inhibitor is talabostat, the OX40 agonist is PF-04518600, and the immune checkpoint inhibitor is pembrolizumab.

In embodiments, the subject with cancer is administered therapeutically effective amounts of a selective dipeptidyl peptidase inhibitor, an OX40 agonist and an immune checkpoint inhibitor, wherein the selective dipeptidyl peptidase inhibitor is talabostat, the OX40 agonist is PF-04518600, and the immune checkpoint inhibitor is nivolumab.

In embodiments, the subject with cancer is administered therapeutically effective amounts of a selective dipeptidyl peptidase inhibitor, an OX40 agonist and an immune checkpoint inhibitor, wherein the selective dipeptidyl peptidase inhibitor is talabostat, the OX40 agonist is PF-04518600, and the immune checkpoint inhibitor is a CTLA4 inhibitor.

In embodiments, the subject with cancer is administered therapeutically effective amounts of a selective dipeptidyl peptidase inhibitor, an OX40 agonist and an immune checkpoint inhibitor, wherein the selective dipeptidyl peptidase inhibitor is talabostat, the OX40 agonist is PF-04518600, and the immune checkpoint inhibitors are pembrolizumab and ipilimumab.

In embodiments, the methods or compositions described herein generate an anti-tumor memory response in a subject with cancer. In embodiments, the methods or compositions described herein stimulate an increase in pro-inflammatory cytokines in a subject with cancer. In embodiments, the methods or compositions described herein increase tumor cell apoptosis in a subject with cancer. In embodiments, the methods or compositions described herein reduce tumor growth in a subject with cancer. In embodiments, the methods or compositions described herein reduce tumor metastasis in a subject with cancer.

In embodiments, the subject has cancer or is at risk for developing cancer. In embodiments, the subject has cancer that may be at an early stage. In embodiments, the subject has cancer that is at a late stage. In embodiments, the cancer is metastatic. In embodiments, the subject has been diagnosed with an advanced solid tumor.

In embodiments, the subject may be a mammal, such as a primate, ungulate (e.g., cow, pig or horse), domestic pet or domestic animal. In some cases, the subject may be a mammal selected from a rabbit, pig, horse, sheep, cow, cat or dog. In embodiments, the subject is a human.

In embodiments, a combination therapy of the invention is administered to a patient who has not been previously treated with a biotherapeutic or chemotherapeutic agent, i.e., is treatment-naive. In other embodiments, the cancer therapy of the present disclosure is administered to a patient who failed to achieve a sustained response after prior therapy with a biotherapeutic or chemotherapeutic agent, i.e., is treatment-experienced.

In embodiments, the subject with cancer was initially treated with talabostat mesylate, platinum-based chemotherapy, an OX40 agonist (e.g., PF-04518600), a PD-1 inhibitor (e.g., nivolumab or pembrolizumab), chemotherapy, a targeted anti-cancer agent, radiation therapy, surgery, fluoropyrimidine-containing therapy, an EGFR inhibitor, an ALK inhibitor, folinic acid, fluorouracil, oxaliplatin, irinotecan, paclitaxel, gemcitabine, carboplatin, cisplatin, doxorubicin, or combinations thereof and the subject was unresponsive to therapy, i.e., the treatment did not reduce tumor growth and/or metastasis.

Therapeutically effective amounts of the methods and compositions described herein may be administered via injection or oral. Other modes of administration are also contemplated, such as pulmonary, nasal, buccal, rectal, sublingual, enteral and transdermal. As used herein, the term “parenteral” includes subcutaneous, intravenous, intra-arterial, intraperitoneal, intracardiac, intrathecal, and intramuscular injection, as well as infusion injections.

In embodiments, the selective dipeptidyl peptidase inhibitor (e.g., talabostat) is administered orally, intravenously, intramuscularly, subcutaneously, topically, rectally, transdermally, intratracheally, vaginally, intraperitoneally, intraorbitally, by implantation, by inhalation, intrathecally, intraventricularly or intranasally. The preferred route of administration is oral. The selective dipeptidyl peptidase inhibitor can be administered to a subject by any route that delivers the selective dipeptidyl peptidase inhibitor to the affected site, either directly or indirectly.

In embodiments, the selective dipeptidyl peptidase inhibitor (e.g., talabostat mesylate), OX40 agonist and one or more immune checkpoint inhibitors are administered by the same route of administration or by two or three different routes of administration, preferably by two different routes of administration (e.g. oral and parenteral).

In embodiments, the OX40 agonist is administered intravenously, intramuscularly, subcutaneously, topically, orally, transdermally, intraperitoneally, intraorbitally, by implantation, by inhalation, intrathecally, intraventricularly, intracistemally, intrarticularly, intracerebral, intracerebroventricularly, or intranasally, vaginally, intraocularly, rectally, preferably intravenously.

In embodiments, the PD-1 axis inhibitor is administered intravenously, intramuscularly, subcutaneously, topically, orally, transdermally, intraperitoneally, intraorbitally, by implantation, by inhalation, intrathecally, intraventricularly, intracistemally, intrarticularly, intracerebral, intracerebroventricularly, or intranasally, vaginally, intraocularly, rectally, preferably intravenously.

In embodiments, the CTLA4 inhibitor is administered intravenously, intramuscularly, subcutaneously, topically, orally, transdermally, intraperitoneally, intraorbitally, by implantation, by inhalation, intrathecally, intraventricularly, intracistemally, intrarticularly, intracerebral, intracerebroventricularly, or intranasally, vaginally, intraocularly, rectally, preferably intravenously.

The length of treatment using the method and compositions described herein may be determined by one skilled in the art (e.g., a physician). Factors that may influence the length of treatment include, but are not limited to, the stage of disease, the mass and sex of the patient, clinical trial guidelines (e.g., those on the fda.gov website), and information on the approved drug label. For example, a suitable period of treatment can be from 1 week to 2 years, 1 week to 22 months, 1 week to 20 months, 1 week to 18 months, 1 week to 16 months, 1 week to 14 months, 1 week to 12 months, 1 week to 10 months, 1 week to 8 months, 1 week to 6 months, 1 week to 4 months 1 week to 2 months, 1 week to 1 month, 2 weeks to 2 years, 2 weeks to 22 months, 2 weeks to 20 months, 2 weeks to 18 months, 2 weeks to 16 months, 2 weeks to 14 months, 2 weeks to 12 months, 2 weeks to 10 months, 2 weeks to 8 months, 2 weeks to 6 months, 2 weeks to 4 months, 2 weeks to 2 months, 2 weeks to 1 month, 1 month to 2 years, 1 month to 22 months, 1 month to 20 months, 1 month to 18 months, 1 month to 16 months, 1 month to 14 months, 1 month to 12 months, 1 month to 10 months, 1 month to 8 months, 1 month to 6 months, 1 month to 4 months, 1 month to 2 months, 2 months to 2 years, 2 months to 22 months, 2 months to 20 months, 2 months to 18 months, 2 months to 16 months, 2 months to 14 months, 2 months to 12 months, 2 months to 10 months, 2 months to 8 months, 2 months to 6 months, 2 months to 4 months, 3 months to 2 years, 3 months to 22 months, 3 months to 20 months, 3 months to 18 months, 3 months to 16 months, 3 months to 14 months, 3 months to 12 months, 3 months to 10 months, 3 months to 8 months, 3 months to 6 months, 4 months to 2 years, 4 months to 22 months, 4 months to 20 months, 4 months to 18 months, 4 months to 16 months, 4 months to 14 months, 4 months to 12 months, 4 months to 10 months, 4 months to 8 months, 4 months to 6 months, 6 months to 2 years, 6 months to 22 months, 6 months to 20 months, 6 months to 18 months, 6 months to 16 months, 6 months to 14 months, 6 months to 12 months, 6 months to 10 months, 6 months to 8 months, 8 months to 2 years, 8 months to 22 months, 8 months to 20 months, 8 months to 18 months, 8 months to 16 months, 8 months to 14 months, 8 months to 12 months, 8 months to 10 months, 10 months to 2 years, 10 months to 22 months, 10 months to 20 months, 10 months to 18 months, 10 months to 16 months, 10 months to 14 months, 10 months to 12 months, 12 months to 2 years, 12 months to 22 months, 12 months to 20 months, 12 months to 18 months, 12 months to 16 months, or 12 months to 14 months. In embodiments, the treatment results in a sustained response in a subject after cessation of the treatment.

In embodiments, the OX40 agonist is administered before administration of the selective dipeptidyl peptidase inhibitor. In embodiments, the selective dipeptidyl peptidase inhibitor is administered before administration of the OX40 agonist. In embodiments, the OX40 agonist and the selective dipeptidyl peptidase inhibitor are administered simultaneously.

In embodiments, the OX40 agonist is administered before administration of the selective dipeptidyl peptidase inhibitor and one or more immune checkpoint inhibitors. In embodiments, the OX40 agonist is administered simultaneously with administration of the selective dipeptidyl peptidase inhibitor and one or more immune checkpoint inhibitors. In embodiments, the OX40 agonist is administered after administration of the selective dipeptidyl peptidase inhibitor and one or more immune checkpoint inhibitors.

In embodiments, the immune checkpoint inhibitor is administered before administration of the selective dipeptidyl peptidase inhibitor and the OX40 agonist. In embodiments, the immune checkpoint inhibitor is administered simultaneously with administration of the selective dipeptidyl peptidase inhibitor and the OX40 agonist. In embodiments, the immune checkpoint inhibitor is administered after administration of the selective dipeptidyl peptidase inhibitor and an OX40 agonist.

In embodiments, the selective dipeptidyl peptidase inhibitor is administered before administration of the immune checkpoint inhibitor, and the OX40 agonist. In embodiments, the selective dipeptidyl peptidase inhibitor is administered simultaneously with administration of the immune checkpoint inhibitor and the OX40 agonist. In embodiments, the selective dipeptidyl peptidase inhibitor is administered after administration of the immune checkpoint inhibitor and the OX40 agonist.

In embodiments, the selective dipeptidyl peptidase inhibitor and the OX40 agonist are co-administered. For example, the selective dipeptidyl peptidase inhibitor and the OX40 agonist are two separate formulations and are administered either simultaneously or sequentially.

In embodiments, the selective dipeptidyl peptidase inhibitor, immune checkpoint inhibitor and OX40 agonist are co-administered. For example, the selective dipeptidyl peptidase inhibitor, OX40 agonist, and one or more immune checkpoint inhibitors are three or more separate formulations and are co-administered either simultaneously or sequentially.

In embodiments, administration of the immune checkpoint inhibitor and OX40 agonist, whether simultaneous or sequential, can be performed according to any number of minutes (e.g., 0-60 minutes), hours (e.g., 0-24 hours), days (e.g., 0-7 days), and/or weeks (e.g., 0-52 weeks), and can be determined by one of skill in the art. Exemplary dosages and dosing intervals can also vary over time (e.g., depending upon the patient's clinical response, side effects, etc.), or during different phases of therapy (induction, treatment, or maintenance).

In embodiments, the selective dipeptidyl peptidase inhibitor is administered daily at a dose from about 50 micrograms to about 2400 micrograms, about 100 micrograms to about 1000 micrograms, about 200 micrograms to about 800 micrograms, about 200 micrograms to about 600 micrograms, about 200 micrograms, about 300 micrograms, about 400 micrograms, about 500 micrograms, about 600 micrograms, about 700 micrograms, about 800 micrograms, about 900 micrograms, about 1000 micrograms, about 1100 micrograms or about 1200 micrograms.

In embodiments, the selective dipeptidyl peptidase inhibitor is talabostat mesylate and is administered daily at a dose from about 50 micrograms to about 2400 micrograms, about 100 micrograms to about 1000 micrograms, about 200 micrograms to about 800 micrograms, about 200 micrograms to about 600 micrograms, about 200 micrograms, about 300 micrograms, about 400 micrograms, about 500 micrograms, about 600 micrograms, about 700 micrograms, about 800 micrograms, about 900 micrograms, about 1000 micrograms, about 1100 micrograms or about 1200 micrograms.

In embodiments, the dosage of the selective dipeptidyl peptidase inhibitor is about 0.001 mg/kg to about 10 mg/kg, about 0.001 mg/kg to about 1 mg/kg, about 0.001 mg/kg to about 0.05 mg/kg, about 0.001 mg/kg to about 0.035 mg/kg, about 0.002 mg/kg to about 5 mg/kg, about 0.002 mg/kg to about 3 mg/kg, about 0.002 mg/kg to about 2 mg/kg, about 0.002 mg/kg to about 0.05 mg/kg, about 0.002 mg/kg to about 0.035 mg/kg, about 0.003 mg/kg to about 2.0 mg/kg, about 0.003 to 0.1 mg/kg, about 0.003 to about 0.05 mg/kg, about 0.003 mg/kg to about 0.035 mg/kg, about 0.004 mg/kg to about 2.5 mg/kg, about 0.004 to about 2 mg/kg, about 0.004 to about 0.1 mg/kg, about 0.004 to about 0.05 mg/kg, about 0.004 mg/kg to about 0.035 mg/kg, about 0.005 mg/kg to about 2.5 mg/kg, about 0.005 to about 2 mg/kg, about 0.005 to about 0.1 mg/kg, about 0.005 to about 0.05 mg/kg, about 0.005 mg/kg to about 0.035 mg/kg, about 0.006 mg/kg to about 2.5 mg/kg, about 0.006 to about 2 mg/kg, about 0.006 to about 0.1 mg/kg, about 0.006 to about 0.05 mg/kg, about 0.006 mg/kg to about 0.035 mg/kg, about 0.007 mg/kg to about 2.5 mg/kg, about 0.007 to about 2 mg/kg, about 0.007 to about 0.1 mg/kg, about 0.007 to about 0.05 mg/kg, about 0.007 mg/kg to about 0.035 mg/kg, about 0.008 mg/kg to about 2.5 mg/kg, about 0.008 to about 2 mg/kg, about 0.008 to about 0.1 mg/kg, about 0.008 to about 0.05 mg/kg, about 0.008 mg/kg to about 0.035 mg/kg, about 0.009 mg/kg to about 2.5 mg/kg, about 0.009 to about 2 mg/kg, about 0.009 to about 0.1 mg/kg, about 0.009 to about 0.05 mg/kg, about 0.009 mg/kg to about 0.035 mg/kg, about 0.010 mg/kg to about 1.5 mg/kg, about 0.010 to about 0.1 mg/kg, about 0.010 to about 0.05 mg/kg, about 0.010 mg/kg to about 0.035 mg/kg, about 0.011 mg/kg to about 1.5 mg/kg, about 0.011 to about 0.1 mg/kg, about 0.011 to about 0.05 mg/kg, about 0.011 mg/kg to about 0.035 mg/kg, about 0.012 mg/kg to about 0.05 mg/kg, about 0.012 mg/kg to about 0.035 mg/kg, about 0.012 mg/kg to about 1.5 mg/kg, about 0.013 mg/kg to about 1 mg/kg, about 0.013 mg/kg to about 0.05 mg/kg or about 0.013 mg/kg to about 0.035 mg/kg.

In certain embodiments, the selective dipeptidyl peptidase inhibitor is talabostat mesylate and is administered at a dose of about 0.001 mg/kg, about 0.002 mg/kg, about 0.003 mg/kg, about 0.004 mg/kg, about 0.005 mg/kg, about 0.006 mg/kg, about 0.007 mg/kg, about 0.008 mg/kg, about 0.009 mg/kg, about 0.010 mg/kg, about 0.011 mg/kg, about 0.012 mg/kg, about 0.013 mg/kg, about 0.014 mg/kg, about 0.020 mg/kg, about 0.025 mg/kg, about 0.030 mg/kg or about 0.035 mg/kg. In preferred embodiments, each dose of talabostat mesylate is administered at about 0.002 mg/kg, about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.011 mg/kg, about 0.012 mg/kg, about 0.013 mg/kg or about 0.014 mg/kg. The dose of talabostat mesylate may vary from about 0.001 mg/kg to about 10 mg/kg, preferably about 0.001 mg/kg to about 3 mg/kg, more preferably about 0.001 mg/kg to about 2 mg/kg. The dose of talabostat mesylate may vary from about 0.001 mg/kg to about 0.05 mg/kg, preferably about 0.001 mg/kg to about 0.035 mg/kg.

In embodiments, the selective dipeptidyl peptidase inhibitor (e.g., talabostat mesylate) is administered at three doses per day, two doses per day, one dose per day, one dose every 2 days, one dose every 3 days, one dose every 4 days, one dose every 5 days, once a week, once every two weeks or once every three weeks or once every four weeks, preferably once a day.

In embodiments, the PD-1 axis inhibitor is administered intravenously at a dose of about 0.01 mg/kg to about 20 mg/kg, about 0.1 mg/kg to about 20 mg/kg, about 1 mg/kg to about 10 mg/kg, about 1 mg/kg to about 5 mg/kg, or about 1 mg/kg to about 3 mg/kg. In embodiments, the PD-1 axis inhibitor is administered intravenously at a dose of about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg or about 20 mg/kg.

In embodiments, the PD-1 axis inhibitor is administered at doses of 50-600 mg, preferably 50 mg, 60 mg, 70 mg, 75 mg, 100 mg, 125 mg, 150 mg, 175 mg, 200 mg, 225 mg, 250 mg, 375 mg, 400 mg, 425 mg, 450 mg, 475 mg, 500 mg, or 600 mg, more preferably 60 mg, 100 mg, 200 mg, 400 mg or 600 mg.

In embodiments, the PD-1 axis inhibitor is administered once per day, once every 2 days, once every 3 days, once every 4 days, once every 5 days, once every week, once every 2 weeks, once every 3 weeks, once every 4 weeks, twice every week, twice every 2 weeks, twice every 3 weeks, or twice every 4 weeks. In embodiments, the PD-1 axis inhibitor is administered twice every 4 weeks.

In embodiments, the anti-PD-1 antibody may be administered intravenously at a dose of about 1 mg/mg to about 40 mg/mg or any of the subranges of the range described herein, e.g., about 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg or 20 mg/kg. These doses may be at intervals of about 14 days (±2 days), about 21 days (±2 days), or about 30 days (±2 days) throughout the course of treatment.

In embodiments, the PD-1 axis inhibitor is nivolumab and is administered intravenously at a dosing regimen of 240 mg Q2W (Q2W=one dose every two weeks), 480 mg Q4W (Q4W=one dose every four weeks), 1 mg/kg Q2W, 2 mg/kg Q2W, 3 mg/kg Q2W, 5 mg/kg Q2W, 10 mg/kg Q2W, 1 mg/kg Q3W (Q3W=one dose every three weeks), 2 mg/kg Q3W, 3 mg/kg Q3W, 5 mg/kg Q3W or 10 mg/kg Q3W.

In embodiments, the PD-1 axis inhibitor is pembrolizumab (MK-3475) and is administered at a dose of 1 mg/kg Q2W, 2 mg/kg Q2W, 3 mg/kg Q2W, 5 mg/kg Q2W, 10 mg/kg Q2W, 1 mg/kg Q3W, 2 mg/kg Q3W, 3 mg/kg Q3W, 5 mg/kg Q3W, 200 mg Q3W or 10 mg/kg Q3W. In embodiments, the dosing regimen of pembrolizumab is 2 mg/kg Q2W or 10 mg/kg Q2W.

In embodiments, the PD-1 axis inhibitor is avelumab, and is administered at a dosing regimen of 800 mg Q2W, 1 mg/kg Q2W, 2 mg/kg Q2W, 3 mg/kg Q2W, 5 mg/kg Q2W, 10 mg/kg Q2W, 20 mg/kg Q2W, 1 mg/kg Q3W, 2 mg/kg Q3W, 3 mg/kg Q3W, 5 mg/kg Q3W or 10 mg/kg Q3W. In embodiments.

In embodiments, the anti-CTLA4 antibody is administered intravenously at a dose of about 1 mg/mg to about 20 mg/mg or any of the subranges of the range described herein, e.g., about 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg or 20 mg/kg. These doses may be at intervals of about 7 days (±2 days), about 14 days (±2 days), about 21 days (±2 days), or about 30 days (±2 days) throughout the course of treatment.

In embodiments, the dose of nivolumab is 3 mg/kg body weight, which is intravenously administered over a period of 60 minutes. In embodiments, the dose of pembrolizumab is 2 mg/kg body, which is intravenously administered over a period of 30 min. In embodiments, the dose of atezolizumab is 1200 mg infused over a period of 60 min. In embodiments, the dose of durvalumab is 10 mg/kg body weight, which is intravenously administered over a period of 60 min.

In embodiments, the CTLA4 inhibitor is ipilimumab, and is intravenously administered at a dose of 1 mg/kg over a period of 30 minutes every 3 weeks for a maximum of 4 doses. In embodiments, ipilimumab is intravenously administered at a dose of 10 mg/kg over a period of 90 minutes every 3 weeks for 4 doses, followed by 10 mg/kg every 12 weeks for up to 3 years, or until disease recurrence or unacceptable toxicity. In embodiments ipilimumab is intravenously administered at a dose of 3 mg/kg over a period of 90 minutes every 3 weeks for a total of 4 doses.

In embodiments, the OX40 agonist may be administered at a dose of about 0.01 mg/kg to about 20 mg/kg, 0.05 mg/kg to about 20 mg/kg, preferably about 0.1 mg/kg to about 15 mg/kg, more preferably about 0.1 mg/kg to about 10 mg/kg, most preferably about 0.1 mg/kg to about 5 mg/kg. In embodiments, the OX40 agonist is administered at a dose of about 0.01 mg/kg, 0.02 mg/kg, 0.03 mg/kg, 0.04 mg/kg, 0.05 mg/kg, about 0.06 mg/kg, about 0.07 mg/kg, about 0.08 mg/kg, about 0.09 mg/kg, about 0.10 mg/kg, about 0.11 mg/kg, about 0.12 mg/kg, about 0.13 mg/kg, about 0.14 mg/kg, about 0.15 mg/kg, about 0.16 mg/kg, about 0.17 mg/kg, about 0.18 mg/kg, about 0.19 mg/kg, about 0.20 mg/kg, about 0.21 mg/kg, about 0.22 mg/kg, about 0.23 mg/kg, about 0.24 mg/kg, 0.25 mg/kg, about 0.26 mg/kg, about 0.27 mg/kg, about 0.28 mg/kg, about 0.29 mg/kg, about 0.30 mg/kg, about 0.31 mg/kg, about 0.32 mg/kg, about 0.33 mg/kg, about 0.34 mg/kg, about 0.35 mg/kg, about 0.36 mg/kg, about 0.37 mg/kg, about 0.38 mg/kg, about 0.39 mg/kg, about 0.40 mg/kg, about 0.41 mg/kg, about 0.42 mg/kg, about 0.43 mg/kg, about 0.44 mg/kg, about 0.45 mg/kg, about 0.46 mg/kg, about 0.47 mg/kg, about 0.48 mg/kg, about 0.49 mg/kg, about 0.50 mg/kg, about 0.51 mg/kg, about 0.52 mg/kg, about 0.53 mg/kg, about 0.54 mg/kg, about 0.55 mg/kg, about 0.56 mg/kg, about 0.57 mg/kg, about 0.58 mg/kg, about 0.59 mg/kg, about 0.60 mg/kg, about 0.61 mg/kg, about 0.62 mg/kg, about 0.62 mg/kg, about 0.65 mg/kg, about 0.70 mg/kg, about 0.75 mg/kg, about 0.80 mg/kg, about 0.83 mg/kg, about 0.85 mg/kg, about 0.90 mg/kg, about 0.95 mg/kg, about 1.00 mg/kg, about 2.00 mg/kg, about 3.00 mg/kg, about 4.00 mg/kg, about 5.00 mg/kg, about 6.00 mg/kg, about 7.00 mg/kg, about 8.00 mg/kg, about 9.00 mg/kg, about 10.00 mg/kg, about 12.00 mg/kg, about 14.00 mg/kg, about 16.00 mg/kg, about 18.00 mg/kg or about 20.00 mg/kg.

In embodiments, the OX40 agonist may be administered at a unit dose of about 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.5 mg, 3 mg, 3.5 mg, 5 mg, about 7 mg, 10.5 mg, 14 mg, 17.5 mg, 21 mg, 24.5 mg, 28 mg, 31.5 mg, 35 mg, 38.5 mg, 42 mg, 45.5 mg, 49 mg, 52.5 mg, 56 mg, 58.1 mg, 59.5 mg, 63 mg, 66.5 mg, 70 mg, 140 mg, 210 mg, 280 mg, 350 mg, 420 mg, 490 mg, 560 mg, 630 mg, 700 mg, 840 mg, 980 mg, 1120 mg, 1260 mg or 1400 mg. The OX40 agonist unit dose range may vary from about 3.5 mg to about 1400 mg, e.g., about 7 mg to about 1050 mg, about 7 mg to about 700 mg or about 7 mg to about 350 mg.

In embodiments, the OX40 agonist may be administered every two weeks at a dose selected from the group consisting of 0.01 mg/kg, 0.03 mg/kg, 0.1 mg/kg, 0.3 mg/kg, 1 mg/kg, 1.5 mg/kg, 3 mg/kg, 5 mg/kg and 10 mg/kg.

In embodiments, the OX40 agonist may be administered at one dose per day, one dose every 2 days, one dose every 3 days, one dose every 4 days, one dose every 5 days, two dose every day, one dose every week, one dose every 2 weeks, one dose every 3 weeks, or one dose every 4 weeks, preferably one dose every 2 weeks.

In embodiments, the OX40 agonist may be administered intravenously at a dose of 0.01 mg/kg Q2W, 0.1 mg/kg Q2W, 0.3 mg/kg Q2W, 1 mg/kg Q2W, 1.5 mg/kg Q2W, 2 mg/kg Q2W, 3 mg/kg Q2W, 5 mg/kg Q2W, 10 mg/kg Q2W, 0.01 mg/kg Q3W, 0.1 mg/kg Q3W, 0.3 mg/kg Q3W, 1 mg/kg Q3W, 1.5 mg/kg Q3W, 2 mg/kg Q3W, 3 mg/kg Q3W, 5 mg/kg Q3W, 10 mg/kg Q3W, 0.01 mg/kg Q4W, 0.1 mg/kg Q4W, 0.3 mg/kg Q4W, 1 mg/kg Q4W, 1.5 mg/kg Q4W, 2 mg/kg Q4W, 3 mg/kg Q4W, 5 mg/kg Q4W, or 10 mg/kg Q4W.

In embodiments, the OX40 agonist is PF04518600 and is intravenously administered at a dose from about 0.01 mg/kg to about 3 mg/kg once every 2 weeks. In embodiments, PF04518600 is intravenously administered at a dose from about 0.01 mg/kg to about 0.1 mg/kg once every 2 weeks.

In embodiments, pogalizumab is intravenously administered at a dose of 300 mg every 3 weeks. In another embodiment, MEDI 0562 is intravenously administered at a dose of 0.03 mg/kg, 0.1 mg/kg, 0.3 mg/kg, 1 mg/kg, 3 mg/kg, or 10 mg/kg every 2 weeks.

In embodiments, the selective dipeptidyl peptidase inhibitor and OX40 agonist is preferably administered for at least 12 weeks (three 4-week cycles or four 3-week cycles), more preferably at least 24 weeks, and even more preferably at least 2 to 4 weeks after the patient achieves a complete response. In embodiments, the selective dipeptidyl peptidase inhibitor, OX40 agonist and one or more immune checkpoint inhibitors is preferably administered for at least 12 weeks (three 4-week cycles or four 3-week cycles), more preferably at least 24 weeks, and even more preferably at least 2 to 4 weeks after the patient achieves a complete response.

In embodiments, a single administration cycle comprises 21 days (21-day cycle). In embodiments, talabostat mesylate is administered orally once daily on Days 1 to 14 of a 21-day cycle and the OX40 agonist (0.01 to 10 mg/kg) is administered intravenously on Day 1 every Q2W.

In embodiments, a single administration cycle comprises 21 days. In embodiments, talabostat mesylate (200 mcg to 600 mcg) is administered once daily on days 1 to 14 of the 21 day cycle, pembrolizumab (200 mcg) is administered intravenously on day 1 of the 21 day cycle and the OX40 agonist (0.01 mg/kg to 10 mg/kg) is administered intravenously on day 1 and day 14 of the 21 day cycle. In another embodiment, talabostat mesylate is administered twice daily at a dose of 300 mcg (600 mcg per day) on days 1 to 14 of the 21 day cycle.

### Treatment Outcomes

Patients treated according to the methods and compositions disclosed herein preferably experience an improvement in cancer prognosis. In embodiments, an improvement in cancer prognosis is a reduction in the quantity and/or size of a measurable tumor. In embodiments, an improvement in cancer prognosis is measured by a reduction in tumor metastasis. In embodiments, an improvement in cancer prognosis is increased survival of the patient with cancer.

In embodiments, the responsiveness to cancer therapy disclosed herein is determined using chest x-rays, computed tomography or magnetic resonance imaging. In embodiments, the responsiveness to the methods and compositions described herein is determined using cytology or histology. In embodiments, the responsiveness to the methods and compositions described herein is determined by showing an extension of progression free survival and/or overall survival.

In embodiments, the methods and compositions provided herein result in a 1% to 99% reduction in tumor volume or growth. For example, In embodiments, the methods and compositions provided herein result in a 1% to 98%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100% reduction in the volume of one or more solid tumors in a patient with cancer.

In embodiments, the methods and compositions described herein can provide for a 1% to 99% reduction in tumor metastasis in a patient with cancer. In embodiments, the methods and compositions described herein can provide a 1% to 98%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100% reduction in tumor metastasis in a patient with cancer.

In embodiments, the methods and compositions described herein can result in an increase of about 1% to 400%, 1% to 380%, 1% to 360%, 1% to 340%, 1% to 320%, 1% to 300%, 1% to 280%, 1% to 260%, 1% to 240%, 1% to 220%, 1% to 200%, 1% to 180%, 1% to 160%, 1% to 140%, 1% to 120%, 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 400%, 5% to 380%, 5% to 360%, 5% to 340%, 5% to 320%, 5% to 300%, 5% to 280%, 5% to 260%, 5% to 240%, 5% to 220%, 5% to 200%, 5% to 180%, 5% to 160%, 5% to 140%, 5% to 120%, 5% to 100%, 5% to 90%, 5% to 80%, 5% to 70%, 5% to 60%, 5% to 50%, 5% to 40%, 5% to 30%, 5% to 20%, 5% to 10%, 10% to 400%, 10% to 380%, 10% to 360%, 10% to 340%, 10% to 320%, 10% to 300%, 10% to 280%, 10% to 260%, 10% to 240%, 10% to 220%, 10% to 200%, 10% to 180%, 10% to 160%, 10% to 140%, 10% to 120%, 10% to 100%, 10% to 90%, 10% to 80%, 10% to 70%, 10% to 60%, 10% to 50%, 10% to 40%, 10% to 30%, 10% to 20%, 20% to 400%, 20% to 380%, 20% to 360%, 20% to 340%, 20% to 320%, 20% to 300%, 20% to 280%, 20% to 260%, 20% to 240%, 20% to 220%, 20% to 200%, 20% to 180%, 20% to 160%, 20% to 140%, 20% to 120%, 20% to 100%, 20% to 90%, 20% to 80%, 20% to 70%, 20% to 60%, 20% to 50%, 20% to 40%, 20% to 30%, 30% to 400%, 30% to 380%, 30% to 360%, 30% to 340%, 30% to 320%, 30% to 300%, 30% to 280%, 30% to 260%, 30% to 240%, 30% to 220%, 30% to 200%, 30% to 180%, 30% to 160%, 30% to 140%, 30% to 120%, 30% to 100%, 30% to 90%, 30% to 80%, 30% to 70%, 30% to 60%, 30% to 50%, 30% to 40%, 40% to 400%, 40% to 380%, 40% to 360%, 40% to 340%, 40% to 320%, 40% to 300%, 40% to 280%, 40% to 260%, 40% to 240%, 40% to 220%, 40% to 200%, 40% to 180%, 40% to 160%, 40% to 140%, 40% to 120%, 40% to 100%, 40% to 90%, 40% to 80%, 40% to 70%, 40% to 60%, 40% to 50%, 50% to 400%, 50% to 380%, 50% to 360%, 50% to 340%, 50% to 320%, 50% to 300%, 50% to 280%, 50% to 260%, 50% to 240%, 50% to 220%, 50% to 200%, 50% to 180%, 50% to 160%, 50% to 140%, 50% to 140%, 50% to 120%, 50% to 100%, 50% to 90%, 50% to 80%, 50% to 70%, 50% to 60%, 60% to 400%, 60% to 380%, 60% to 360%, 60% to 340%, 60% to 320%, 60% to 300%, 60% to 280%, 60% to 260%, 60% to 240%, 60% to 220%, 60% to 200%, 60% to 180%, 60% to 160%, 60% to 140%, 60% to 120%, 60% to 100%, 60% to 90%, 60% to 80%, 60% to 70%, 70% to 400%, 70% to 380%, 70% to 360%, 70% to 340%, 70% to 320%, 70% to 300%, 70% to 280%, 70% to 260%, 70% to 240%, 70% to 220%, 70% to 200%, 70% to 180%, 70% to 160%, 70% to 140%, 70% to 120%, to 100%, 70% to 90%, 70% to 80%, 80% to 400%, 80% to 380%, 80% to 360%, 80% to 340%, 80% to 320%, 80% to 300%, 80% to 280%, 80% to 260%, 80% to 240%, 80% to 220%, 80% to 200%, 80% to 180%, 80% to 160%, 80% to 140%, 80% to 120%, 80% to 100%, 80% to 90%, 90% to 400%, 90% to 380%, 90% to 360%, 90% to 340%, 90% to 320%, 90% to 300%, 90% to 280%, 90% to 260%, 90% to 240%, 90% to 220%, 90% to 200%, 90% to 180%, 90% to 160%, 90% to 140%, 90% to 120%, 90% to 100%, 100% to 400%, 100% to 380%, 100% to 360%, 100% to 340%, 100% to 320%, 100% to 300%, 100% to 280%, 100% to 260%, 100% to 240%, 100% to 220%, 100% to 200%, 100% to 180%, 100% to 160%, 100% to 140%, 100% to 120%, 120% to 400%, 120% to 380%, 120% to 360%, 120% to 340%, 120% to 320%, 120% to 300%, 120% to 280%, 120% to 260%, 120% to 240%, 120% to 220%, 120% to 200%, 120% to 180%, 120% to 160%, 120% to 140%, 140% to 400%, 140% to 380%, 140% to 360%, 140% to 340%, 140% to 320%, 140% to 300%, 140% to 280%, 140% to 260%, 140% to 240%, 140% to 220%, 140% to 200%, 140% to 180%, 140% to 160%, 160% to 400%, 160% to 380%, 160% to 360%, 160% to 340%, 160% to 320%, 160% to 300%, 160% to 280%, 160% to 260%, 160% to 240%, 160% to 220%, 160% to 200%, 160% to 180%, 180% to 400%, 180% to 380%, 180% to 360%, 180% to 340%, 180% to 320%, 180% to 300%, 180% to 280%, 180% to 260%, 180% to 240%, 180% to 220%, 180% to 200%, 200% to 400%, 200% to 380%, 200% to 360%, 200% to 340%, 200% to 320%, 200% to 300%, 200% to 280%, 200% to 260%, 200% to 240%, 200% to 220%, 220% to 400%, 220% to 380%, 220% to 360%, 220% to 340%, 220% to 320%, 220% to 300%, 220% to 280%, 220% to 260%, 220% to 240%, 240% to 400%, 240% to 380%, 240% to 360%, 240% to 340%, 240% to 320%, 240% to 300%, 240% to 280%, 240% to 260%, 260% to 400%, 260% to 380%, 260% to 360%, 260% to 340%, 260% to 320%, 260% to 300%, 260% to 280%, 280% to 400%, 280% to 380%, 280% to 360%, 280% to 340%, 280% to 320%, 280% to 300%, 300% to 400%, 300% to 380%, 300% to 360%, 300% to 340%, or 300% to 320% in the time of survival in a patient with cancer.

In embodiments, the cancer patient treated with the methods and compositions disclosed herein exhibits a complete response, a partial response or stable disease.

In embodiments, the methods and compositions disclosed herein result in tumor shrinkage and/or a decrease in the growth rate of the tumor. In embodiments, the rate of tumor cell proliferation is inhibited. In yet another embodiment, one or more of the following can occur: the number of cancer cells can be reduced, cancer cell infiltration into peripheral organs can be inhibited or reduced, tumor metastasis can be reduced or inhibited, recurrence of the tumor can be prevented or delayed, or one or more of the symptoms associated with cancer can be reduced or eliminated.

In embodiments, combination of the selective dipeptidyl peptidase inhibitor, OX40 agonist and immune checkpoint inhibitor produce a comparable clinical benefit rate (clinical benefit rate=complete remission+partial remission+stable disease) better than that achieved by treatment with the agents alone. In embodiments, combination of the selective dipeptidyl peptidase inhibitor, OX40 agonist and immune checkpoint inhibitor produce an improved clinical benefit rate of about 20%, 30%, 40%, 50%, 60%, 70%, 80% or more compared to treatment with the agents alone.

In embodiments, the CD8+ T cells in the individual have enhanced priming, activation, proliferation and/or cytolytic activity upon treatment with the selective dipeptidyl peptidase inhibitor, OX40 agonist and one or more immune checkpoint inhibitors compared to treatment with the agents alone.

In embodiments, the number of CD4+ and/or CD8+ T cells is elevated in the cancer subject upon administration of the selective dipeptidyl peptidase inhibitor, OX-40 agonist and one or more immune checkpoint inhibitors. In embodiments, the CD4+ and/or CD8+ T cells are activated in the cancer subject upon administration of the selective dipeptidyl peptidase inhibitor, OX-40 agonist and immune checkpoint inhibitor. Activation of CD4+ and/or CD8+ T cells is characterized by IFNγ production and/or enhanced cytolytic activity. In embodiments, the CD4+ and/or CD8+ T cells in the cancer subject exhibit an increase in cytokine production upon administration of the selective dipeptidyl peptidase inhibitor, OX-40 agonist and immune checkpoint inhibitor. Cytokines that may be increased include, but are not limited to, G-CSF, MCP-1, Eotaxin, IFN-γ, KC, TNF-α, IL-5, IL-6, IL-1β, IL-12p70 and IL-18.

In embodiments, the CD4+ and/or CD8+ T cell is an effector memory T cell. In embodiments, the CD4+ and/or CD8+ effector memory T cell increases production of IFNγ and/or enhances cytolytic activity upon administration of the selective dipeptidyl peptidase inhibitor, OX-40 agonist and one or more immune checkpoint inhibitor described herein.

In embodiments, serum levels of the cytokine IL-18 and/or the chemokines GM-CSF or G-CSF are increased in the cancer subject upon treatment with the selective dipeptidyl peptidase inhibitor, OX-40 agonist and immune checkpoint inhibitor compared to treatment with the agent alone.

### Pharmaceutical Compositions

In some embodiments, the present disclosure provides a pharmaceutical composition comprising a selective dipeptidyl peptidase inhibitor and an OX40 agonist together with one or more pharmaceutically acceptable carriers and/or excipients.

In embodiments, the present disclosure provides a pharmaceutical composition comprising a selective dipeptidyl peptidase inhibitor, an OX40 agonist and an immune checkpoint inhibitor together with one or more pharmaceutically acceptable carriers and/or excipients.

In other embodiments, the present disclosure provides two separate pharmaceutical compositions, namely (1) a pharmaceutical composition comprising a selective dipeptidyl peptidase inhibitor together with one or more pharmaceutically acceptable carriers and/or excipients and (2) a pharmaceutical composition comprising an OX40 agonist together with one or more pharmaceutically acceptable carriers and/or excipients. The compositions may be administered to the subject at the same time, sequentially in any suitable order or separately (including intermittently), such that the combination therapy provides an effective treatment of cancer in said subject.

In other embodiments, the present disclosure provides three separate pharmaceutical compositions, namely (1) a pharmaceutical composition comprising a selective dipeptidyl peptidase inhibitor together with one or more pharmaceutically acceptable carriers and/or excipients (2) a pharmaceutical composition comprising an OX40 agonist together with one or more pharmaceutically acceptable carriers and/or excipients and (3) a pharmaceutical composition comprising an immune checkpoint inhibitor together with one or more pharmaceutically acceptable carriers and/or excipients. The compositions may be administered to the subject at the same time, sequentially in any suitable order or separately (including intermittently), such that the combination therapy provides an effective treatment of cancer in said subject.

In other aspects, the present disclosure provides two separate pharmaceutical compositions, namely (1) a pharmaceutical composition comprising a selective dipeptidyl peptidase inhibitor and an immune checkpoint inhibitor together with one or more pharmaceutically acceptable carriers and/or excipients and (2) a pharmaceutical composition comprising an OX40 agonist together with one or more pharmaceutically acceptable carriers and/or excipients, or (1) a pharmaceutical composition comprising a selective dipeptidyl peptidase inhibitor together with one or more pharmaceutically acceptable carriers and/or excipients and (2) a pharmaceutical composition comprising an immune checkpoint inhibitor and OX40 agonist together with one or more pharmaceutically acceptable carriers and/or excipients, or (1) a pharmaceutical composition comprising a selective dipeptidyl peptidase inhibitor and an OX40 agonist together with one or more pharmaceutically acceptable carriers and/or excipients and (2) a pharmaceutical composition comprising an immune checkpoint inhibitor together with one or more pharmaceutically acceptable carriers and/or excipients. The compositions may be administered to the subject at the same time, sequentially in any suitable order or separately (including intermittently), such that the combination therapy provides an effective treatment of cancer in said subject.

In some embodiments, the pharmaceutical composition comprises a selective dipeptidyl peptidase inhibitor, wherein the selective dipeptidyl peptidase inhibitor is talabostat or a pharmaceutically acceptable salt thereof (e.g., talabostat mesylate) in the form of a tablet.

In embodiments, the pharmaceutical composition comprises a PD-1 axis inhibitor, wherein the PD-1 axis inhibitor is an antibody (e.g., pembrolizumab or nivolumab) in liquid formulation. The formulation may contain one or more of mannitol, sucrose, pentetic acid, sodium chloride, sodium citrate, histidine, and polysorbate 80. In embodiments, the pharmaceutical composition is the PD-1 axis inhibitor nivolumab and each mL comprises nivolumab (e.g., 10 mg), mannitol (e.g., 30 mg), pentetic acid (e.g., 0.008 mg), polysorbate 80 (e.g., 0.2 mg), sodium chloride (e.g., 2.92 mg), sodium citrate dihydrate (e.g., 5.88 mg), and water for injection, USP. The formulation may be adjusted to about pH 6 using an acid or base, for example, hydrochloric acid or sodium hydroxide. In embodiments, the pharmaceutical composition is the PD-1 axis inhibitor pembrolizumab and comprises 25 mg/mL MK-3475, 7% (w/v) sucrose, 0.02% (w/v) polysorbate 80 in 10 mM histidine buffer, pH 5.5. In embodiments, the pharmaceutical composition is the PD-1 axis inhibitor avelumab and comprises 20 mg avelumab, D-mannitol (51 mg), glacial acetic acid (0.6 mg), polysorbate 20 (0.5 mg), sodium hydroxide (0.3 mg), and water for injection. The pH range of the solution is 5.0-5.6.

In embodiments, the pharmaceutical composition comprises a CTLA4 inhibitor, wherein the CTLA4 inhibitor is an antibody (e.g., ipilimumab) in liquid formulation. The formulation may contain one or more of diethylene triamine pentaacetic acid (DTPA), mannitol, polysorbate 80 (vegetable origin), sodium chloride, tris hydrochloride, and water for injection. In embodiments, the pharmaceutical composition is the CTLA4 inhibitor ipilimumab and each mL comprises 5 mg of ipilimumab and the following inactive ingredients: DTPA (0.04 mg), mannitol (10 mg), polysorbate 80 (vegetable origin, 0.1 mg), sodium chloride (5.85 mg), tris hydrochloride (3.15 mg), and water for injection. The pH of the solution is 7.

In embodiments, the pharmaceutical composition comprises an OX40 agonist, wherein the OX40 agonist is an antibody, such as PF-04518600, in the form of a liquid.

Each active component can be administered separately. Alternatively, if administration of two active components (e.g., OX40 agonist and an immune checkpoint inhibitor) is desired to be simultaneous and the two active components are compatible together in a given formulation then simultaneous administration can be achieved via administration of a single dosage formulation (e.g., intravenous administration of a formulation that contains the pharmacologically active agents). One of ordinary skill in the art can determine through routine testing whether two given pharmacological agents are compatible together in a given formulation.

The pharmaceutical compositions may be formulated in a variety of ways, including for example, liquid, semi-solid and solid dosage forms, such as liquid solutions (e.g., injectable and infusible solutions), dispersions or suspensions, tablets, pills, powders, liposomes and suppositories. In embodiments, the compositions may be formulated as injectable or infusible solutions. Liquid formulations can be aqueous isotonic solutions or suspensions, and suppositories can be prepared from fatty emulsions or suspensions. The composition may be formulated as an immediate, controlled, extended or delayed release composition.

In embodiments, the pharmaceutical composition (e.g., talabostat or a pharmaceutically acceptable salt thereof) may be administered orally. In other embodiments, the composition of the invention (e.g., a PD-1 axis inhibitor) may be administered parenterally (e.g., intravenously, subcutaneously, intraperitoneally or intramuscularly).

Liquid pharmaceutical compositions for parenteral administration may be formulated for administration by injection or continuous infusion. Routes of administration by injection or infusion can include, but are not limited to, intravenous, intraperitoneal, intramuscular, intrathecal, and subcutaneous. In embodiments, parenteral formulations can include prefilled syringes, vials, powder for infusion for reconstitution, concentrate for infusion to be diluted before delivery (ready to dilute) and solutions (ready to use).

Pharmaceutically acceptable excipients, as used herein, includes, but are not limited to, any and all solvents, dispersion media, or other liquid vehicles, dispersion or suspension aids, diluents, granulating and/or dispersing agents, surface active agents, isotonic agents, thickening or emulsifying agents, preservatives, binders, lubricants or oil, colouring, sweetening or flavouring agents, stabilizers, antioxidants, antimicrobial or antifungal agents, osmolality adjusting agents, pH adjusting agents, buffers, chelates, cryoprotectants, and/or bulking agents, as suited to the particular dosage form desired. Various excipients for formulating pharmaceutical compositions and techniques for preparing the compositions are known in the art (See, Remington: The Science and Practice of Pharmacy, 21st Ed, Lippincott, Williams & Wilkins, 2006; incorporated by reference in its entirety).

Pharmaceutically acceptable carriers include water; saline; phosphate buffered saline; dextrose; glycerol; alcohols such as ethanol and isopropanol; phosphate, citrate and other organic acids; ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; EDTA; salt forming counterions such as sodium; and/or nonionic surfactants such as TWEEN, polyethylene glycol (PEG), and PLURONICS; isotonic agents such as sugars, polyalcohols such as mannitol and sorbitol, and sodium chloride; as well as combinations thereof. Antibacterial and antifungal agents include parabens, chlorobutanol, phenol, ascorbic acid and thimerosal.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases or the like.

Pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, liquid polyethylene glycol, or the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In embodiments, the composition includes isotonic agents, for example, sugars, polyalcohols, such as mannitol, sorbitol, or sodium chloride. Prolonged absorption of the injectable compositions can be brought about by including in the composition an agent which delays absorption, for example, aluminum monostearate and gelatin.

Sterile injectable solutions can be prepared by incorporating the molecule, by itself or in combination with other active agents, in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, one method of preparation is vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Such articles of manufacture will preferably have labels or package inserts indicating that the associated compositions are useful for treating a subject suffering from or predisposed to autoimmune or neoplastic disorders. The pharmaceutical compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compositions or compounds may also be administered as a bolus, electuary or paste. Oral compositions generally include an inert carrier (for example, diluent) or an edible carrier. They can be enclosed in gelatin capsules or compressed into tablets. For oral administration, the therapeutic agents can be combined with carriers and used in the form of tablets, troches, or capsules. Pharmaceutically compatible binding agents, and/or adjuvant materials can be included as part of the composition. The tablets, pills, capsules, troches, and the like can contain any of the following ingredients, or compounds of a similar nature; a binder such as microcrystalline cellulose, gum tragacanth or gelatin; an excipient such as starch or lactose, a disintegrating agent such as alginic acid, primogel, or corn starch; a lubricant such as magnesium stearate or stearates; a glidant such as colloidal silicon dioxide; a sweetening agent such as sucrose or saccharin; or a flavouring agent such as peppermint, methyl salicylate, or orange flavouring. Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Various methods can be used for manufacturing tablets. More particularly, the process may include dissolving talabostat mesylate in a suitable solvent (with or without binder) and this solution is distributed uniformly all over filler particles (may contain other materials) to form agglomerated particles/granules. Wet granulation or coating or spraying process can also be used. Obtained granules are appropriately sized or the granules can be further processed by dry granulation/slugging/roller compaction method followed by milling step to achieve suitable granules of specific particle size distribution. The sized granules are further blended with other components and/or and then lubricated in a suitable blender and compressed into tablets of specific dimensions using appropriate tooling. The coating can be done with appropriate equipment.

In certain embodiments, the pharmaceutical compositions of the present disclosure include biodegradable subcutaneous implant, osmotically controlled device, subcutaneous implant, subcutaneous sustained release injection, lipid nanoparticles, liposomes, and the like. Liquid preparations can include, but are not limited to, solutions, suspensions and emulsions. Such preparations are exemplified by water or water/propylene glycol solutions for parenteral injection. Liquid preparations may also include solutions for intranasal administration.

Aerosol preparations suitable for inhalation may include solutions and solids in powder forms, which may be in combination with a pharmaceutically acceptable carrier, such as an inert compressed gas. Also included are solid preparations which are intended for conversion, shortly before use, to liquid preparations for either oral or parenteral administration. Such liquid forms include solutions, suspensions, and emulsions.

Besides inert diluents, the oral compositions can also include adjuvants such as wetting agents, emulsifying and suspending agents, sweetening, flavoring, coloring, perfuming and preservative agents

Suspensions, in addition to the active compounds, may contain suspending agents as, for example, ethoxylated isostearyl alcohols, polyoxyethylene sorbitol and sorbitan esters, microcrystalline cellulose, aluminium metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminium hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel. Liquid preparations may also include solutions for intranasal administration. Aerosol preparations suitable for inhalation may include solutions and solids in powder form, which may be in combination with a pharmaceutically acceptable carrier, such as an inert compressed gas.

The amount of selective dipeptidyl peptidase inhibitor (e.g., talabostat) present in a composition should, in general, be in the range of about 0.01 to about 30% w/w and preferably in an amount of 0.5 to 20% w/w of the composition. Similarly, the amount of an immune checkpoint inhibitor present in a composition is in the range of about 0.01 to about 30% w/w, preferably in an amount of 0.5 to 20% w/w of the composition. The immune checkpoint inhibitor is selected from the group consisting of a PD-1 inhibitor, a PD-L1 inhibitor, a PD-L2 inhibitor, and a CTLA4 inhibitor.

The precise dose to be employed in the formulation will also depend on the route of administration, and the seriousness of the cancer and should be decided according to the judgment of the practitioner and each patient's circumstances. Effective doses may be extrapolated from dose-response curves derived from in vitro or animal model systems.

In embodiments, the selective dipeptidyl peptidase inhibitor described herein is a formulation comprising an effective amount of a selective dipeptidyl peptidase inhibitor and one or more pharmaceutically acceptable carrier(s) or adjuvant(s) selected from the group comprising bulking agent, buffer, surfactant, pH modifier and the formulation has an appropriate pH.

In embodiments, the selective dipeptidyl peptidase inhibitor described herein is a formulation comprising an effective amount of a selective dipeptidyl peptidase inhibitor (for example, talabostat), and one or more pharmaceutically acceptable carrier(s) or adjuvant(s) selected from the group consisting of a diluent, binder, disintegrant, and glidant.

In another embodiment, the present disclosure relates to a pharmaceutical composition of talabostat for oral administration. In some embodiments, talabostat is formulated as an oral tablet. The pharmaceutical tablet may be an immediate release or a modified release tablet. The tablet may be in the form of matrix or coated form. An exemplary immediate release tablet comprises an effective amount of talabostat and a pharmaceutically-acceptable carrier selected from the group consisting of diluents, binders, disintegrants, glidants, lubricants, pH modifying agents and combinations thereof.

In embodiments, the amount of talabostat in a unit dose is about 100 micrograms per tablet, about 200 micrograms per tablet, about 300 micrograms per tablet, about 400 micrograms per tablet, about 500 micrograms per tablet, about 600 micrograms per tablet, about 700 micrograms per tablet, or about 800 micrograms per tablet.

In embodiments, one or more diluents comprise, but are not limited to, dibasic calcium phosphate, pullulan, maltodextrin, isomalt, sugar pellets, mannitol, spray-dried mannitol, microcrystalline cellulose, dibasic calcium phosphate dihydrate, lactose, sugars, sorbitol, mixture of microcrystalline cellulose and guar gum (Avicel CE-15), mixture of mannitol, polyplasdone and syloid (Pharmaburst), mixture of mannitol, crospovidone and polyvinyl acetate (Ludiflash), isomalt, Panexcea, F-Melt, sucrose, calcium salts and similar inorganic salts, heavy magnesium carbonate and the like, and the mixtures thereof. Preferably, the diluent is lactose or microcrystalline cellulose.

In embodiments, one or more binders comprise, but are not limited to, low-substituted hydroxypropyl cellulose, xanthan gum, polyvinylpyrrolidone (povidone), gelatin, sugars, glucose, natural gums, gums, synthetic celluloses, polymethacrylate, hydroxypropyl methylcellulose, hydroxypropyl cellulose, carboxymethyl cellulose, methyl cellulose, and other cellulose derivatives and the like, and combinations thereof. Preferably, the binder is polyvinylpyrrolidone or hydroxypropyl cellulose or hydroxypropyl methylcellulose.

In embodiments, one or more disintegrants comprise, but are not limited to, at least one or a mixture of sodium starch glycolate, croscarmellose sodium, crospovidone, sodium alginate, gums, starch, and magnesium aluminium silicate. Preferably, the disintegrant is sodium starch glycolate.

In embodiments, one or lubricants comprise, but are not limited to sodium stearyl fumarate, sodium lauryl sulphate, magnesium stearate, polyethylene glycol, metal stearates, hydrogenated castor oil and the like, and the mixtures thereof. Preferably, the lubricant is magnesium stearate.

In embodiments, one or glidants comprise, but are not limited to, stearic acid, colloidal silicon dioxide, talc, aluminium silicate and the like, and the mixtures thereof. Preferably, the glidant is talc.

In embodiments, one or more pH modifying agents comprise, but are not limited to, organic acid, or its salts like phosphoric acid, citric acid and the like.

In embodiments, talabostat is formulated as a tablet and the formulation is shown in Table 1 below.

TABLE 1
Talabostat Formulation

| Formulation Content | Amount (w/w %) |
|---|---|
| Talabostat as a API (available as | 0.01-2 |
| talabostat mesylate) | |
| Binder | 1-50 |
| Disintegrant | 1-15 |
| Lubricant | 0.1-5 |
| Diluent | 30-98 |
| pH modifying agent | 0-15 |

In embodiments, talabostat is formulated as an immediate release tablet and the formulation is shown in Table 2 below.

TABLE 2
Talabostat Immediate Release Formulation

| | Preferred ranges | Amount |
|---|---|---|
| Formulation Content | (w/w %) | (w/w %) |
| Talabostat as a API (available as | 0.01-2 | 0.145 |
| talabostat mesylate) | | |
| Polyvinyl pyrrolidone or | 1-50 | 1.00 |
| hydroxypropylcellulose or | | |
| hydroxypropylmethylcellulose or | | |
| pregelatinized starch as a binder | | |
| Sodium starch glycolate or | 1-15 | 2.5 |
| crospovidone as a disintegrant | | |
| Stearic acid as a lubricant | 0.1-5 | 1.5 |
| Lactose as a diluent | 30-90 | 85.315 |
| Microcrystalline cellulose as a | 5-20 | 9.480 |
| diluent | | |
| Sodium phosphate monobasic, | 0-15 | 0.060 |
| monohydrate as a pH modifying agent | | |
| Phosphoric acid as a pH modifying | For pH | For pH |
| agent | adjustment | adjustment |

In some embodiments, talabostat is formulated as a modified release matrix tablet. A modified release tablet comprises immediate release core and coating wherein said coating comprises modified release material and other pharmaceutical excipients.

Modified release material include, but are not limited to, polyvinyl pyrrolidone (K90), hydroxypropylmethylcellulose (K4M, K10), hydroxypropylcellulose (high viscosity grade), carnauba wax, glyceryl behenate, castor wax, polyvinyl acetate, carboxymethyl ethyl cellulose, ethylcellulose, cellulose phthalates or succinates, in particular cellulose acetate phthalate and hydroxypropylmethylcellulose phthalate, hydroxypropylmethylcellulose succinate or hydroxypropylmethylcellulose acetate succinate; high molecular polyalkylene oxides such as polyethylene oxide and polypropylene oxide and copolymers of ethylene oxide and propylene oxide and the like. Preferably, the modified release material is polyvinyl pyrrolidone (K90), hydroxypropylmethylcellulose (K4M, K10), or hydroxypropylcellulose (high viscosity grade-HF), polyethylene oxide and the like.

The talabostat formulation for the modified release tablet is shown in Table 3 below.

TABLE 3
Talabostat Modified Release Formulation

| Formulation content | Preferred ranges (w/w %) |
|---|---|
| Talabostat as a API (available as Talabostat | 0.01-2 |
| mesylate) | |
| Polyvinyl pyrrolidone (K90) or | 10-50 |
| hydroxypropylcellulose (K4M, K10) or | |
| hydroxypropylmethylcellulose (high viscosity | |
| grade- HF) or pregelatinized starch as a | |
| modified release material | |
| Sodium starch glycolate or crospovidone as a | 0-10 |
| disintegrant | |
| Magnesium stearate or stearic acid as a | 0.1-10 |
| lubricant | |
| Lactose as a diluent | 30-90 |
| Citric acid or phosphoric acid as a pH | 0-15 |
| modifying agent | |

Thus, the disclosure provides a pharmaceutical tablet comprising particles consisting essentially of a talabostat, diluent (e.g., lactose monohydrate) and optionally binder. The particles may be blended with one or more of a binder, a lubricant and a disintegrant and then compressed.

Various methods can be used for manufacturing the tablets of the present disclosure. Preferably, the process includes dissolving talabostat in a suitable solvent (with or without binder) and this solution is distributed uniformly all over filler particles (may contain other materials) to form agglomerated particles/granules. Wet granulation or coating or spraying process can be used for the same. Obtained granules are sized as per the requirement or the granules can be further processed by dry granulation/slugging/roller compaction method followed by milling step to achieve suitable granules of specific particle size distribution. The sized granules are further blended with other components and then lubricated in a suitable blender and compressed into tablets of specific dimensions using appropriate tooling. The coating can be done with appropriate equipment.

### Kits

The present disclosure provides a kit comprising therapeutically effective amounts of a selective dipeptidyl peptidase inhibitor (e.g., talabostat mesylate), an OX40 agonist (e.g., PF-04518600), and an immune checkpoint inhibitor (e.g., PD-1 axis inhibitor).

In embodiments, the kit includes a formulation of a selective dipeptidyl peptidase inhibitor (e.g., talabostat mesylate), an OX40 agonist (e.g., PF-04518600) and an immune checkpoint inhibitor (e.g., PD-1 inhibitor). The therapeutic compositions provided in the kit can be manufactured by the same manufacturer or different manufacturers. Thus, the therapeutic compositions provided in the kit may be separate pharmaceutical compositions that are sold independently of one other. In embodiments, the kit also comprises instructions for the use of the biological agents provided in the kit.

### Cancer Types

Exemplary cancers that may be treated by the methods and compositions of this disclosure include, but are not limited to, pancreatic cancer, colorectal cancer, prostate cancer, skin cancer, colon cancer, ovarian cancer, lung cancer, breast cancer, glioblastoma, gastric cancer, astroglial cancer, neuro-ectodermal cancer, head and neck cancer, triple negative breast cancer, hepatocellular carcinoma, gastroesophageal cancer, hematopoietic cancer, and non-small cell lung cancer.

In embodiments, the cancer is colorectal cancer. In embodiments, the cancer is pancreatic cancer. In other embodiments, the cancer is prostate cancer.

In embodiments, the method of treating colorectal cancer in a subject comprises administering to the subject a therapeutically effective amount of a selective dipeptidyl peptidase inhibitor and a therapeutically effective amount of an OX40 agonist.

In embodiments, the method of treating prostate cancer in a subject comprises administering to the subject a therapeutically effective amount of a selective dipeptidyl peptidase inhibitor and a therapeutically effective amount of an OX40 agonist.

In embodiments, the method of treating colorectal cancer in a subject comprises administering to the subject a therapeutically effective amount of a dipeptidyl peptidase inhibitor, a therapeutically effective amount of an OX40 agonist and a therapeutically effective amount of an immune checkpoint inhibitor.

In embodiments, the method of treating prostate cancer in a subject comprises administering to the subject a therapeutically effective amount of a selective dipeptidyl peptidase inhibitor, a therapeutically effective amount of an OX40 agonist and a therapeutically effective amount of an immune checkpoint inhibitor.

Types of cancers that are inhibited using a selective dipeptidyl peptidase inhibitor (e.g., talabostat mesylate), and an OX40 agonist include, but are not limited to, malignant melanoma, non-small cell lung cancer, renal cancer, monocyte chemotactic protein-1 inhibitor, Hodgkin's disease, gastric cancer, glioblastoma; head and neck cancer, hepatocellular carcinoma, multiple myeloma, oesophageal cancer, small cell lung cancer, urogenital cancer, acute myeloid leukemia, breast cancer, chronic lymphocytic leukemia, diffuse large B cell lymphoma, follicular lymphoma; myelodysplastic syndromes; ovarian cancer; uveal melanoma, colorectal cancer, hematological malignancies, non-Hodgkin's lymphoma, chronic myeloid leukemia and glioma. Additionally, the present disclosure includes refractory or recurrent malignancies whose growth may be inhibited using the therapeutic agents of the present disclosure.

Types of cancers that are inhibited using a selective dipeptidyl peptidase inhibitor (e.g., talabostat mesylate) and an OX40 agonist, include, but are not limited to, melanoma (e.g., metastatic malignant melanoma), hepatocellular carcinoma, head and neck cancer, renal cancer (e.g., clear cell carcinoma), prostate cancer (e.g., hormone refractory prostate adenocarcinoma), breast cancer, glioblastoma, colon cancer and lung cancer (e.g., non-small cell lung cancer, small cell lung cancer), gastric cancer, myelodysplastic syndromes, colorectal cancer, oesophageal cancer, ovarian cancer, urogenital cancer, uveal melanoma, adrenal cancer and liver cancer.

Types of cancers that are inhibited using a selective dipeptidyl peptidase inhibitor (e.g., talabostat mesylate), an OX40 agonist and a PD-1 axis inhibitor, include, but are not limited to, melanoma (e.g., metastatic malignant melanoma), hepatocellular carcinoma, head and neck cancer, renal cancer (e.g., clear cell carcinoma), prostate cancer (e.g., hormone refractory prostate adenocarcinoma), breast cancer, glioblastoma, colon cancer and lung cancer (e.g., non-small cell lung cancer, small cell lung cancer), gastric cancer, myelodysplastic syndromes, colorectal cancer, oesophageal cancer, ovarian cancer, urogenital cancer, uveal melanoma, adrenal cancer and liver cancer.

Types of cancers that are inhibited using a selective dipeptidyl peptidase inhibitor (e.g., talabostat mesylate), an OX40 agonist and a PD-1 inhibitor include, but are not limited to, malignant melanoma, non-small cell lung cancer, renal cancer, monocyte chemotactic protein-1 inhibitor, Hodgkin's disease, gastric cancer, glioblastoma; head and neck cancer, hepatocellular carcinoma, multiple myeloma, oesophageal cancer, small cell lung cancer, urogenital cancer, acute myeloid leukemia, breast cancer, chronic lymphocytic leukemia, diffuse large B cell lymphoma, follicular lymphoma; myelodysplastic syndromes; ovarian cancer; uveal melanoma, colorectal cancer, hematological malignancies, non-Hodgkin's lymphoma, chronic myeloid leukemia and glioma. Additionally, the present disclosure includes refractory or recurrent malignancies whose growth may be inhibited using the therapeutic agents of the present disclosure.

In embodiments, the cancer is a solid tumor. In embodiments, the cancer is urogenital cancer (such as prostate cancer, renal cell cancer or bladder cancer), thyroid cancer, testicular cancer, vulvar cancer, wilm's tumor, hormone sensitive or hormone refractory prostate cancer, gynecological cancers (such as ovarian cancer, cervical cancer, endometrial cancer or uterine cancer), lung cancer, non-small cell lung cancer, small cell lung cancer, gastrointestinal stromal cancers, gastrointestinal cancers (such as non-metastatic or metastatic colorectal cancer, pancreatic cancer, gastric cancer, oesophageal cancer, hepatocellular cancer, cholangiocellular cancer), malignant glioblastoma, malignant mesothelioma, non-metastatic or metastatic breast cancer (such as hormone refractory metastatic breast cancer, triple negative breast cancer), malignant melanoma, melanoma, metastatic melanoma, merkel cell carcinoma or bone and soft tissue sarcomas, oral squamous cell carcinoma, glioblastoma, brain cancer, osteosarcoma, neuroblastoma, advanced metastatic, an inflammatory myofibroblastic tumor (IMT), cholangiocarcinoma, cystadenocarcionoma, ameloblastoma, chondrosarcoma, dermatofibrosarcoma, ganglioglioma, leiomyosarcoma, medulloblastoma, osteoblastoma and inoperable non-inflammatory locally advanced disease and the like. The most preferred cancer is solid tumor (such as pancreatic cancer, colorectal cancer, colon cancer, ovarian cancer, lung cancer, breast cancer, glioblastoma, gastric cancer, skin cancer, prostate cancer, fibrosarcoma, sarcoma, astroglial, neuroectodermal tumors, head and neck cancer, triple negative breast cancer, hepatocellular carcinoma, gastroesophageal cancer, non-small cell lung cancer and the like) or hematopoietic cancer (leukemia, lymphoma, lymphocytic leukemia, non-hodgkin's lymphoma, Hodgkin's lymphoma, an anaplastic large-cell lymphoma, myeloid leukemia, multiple myeloma, acute lymphoblastic leukemia, chronic myeloid leukemia, chronic lymphocytic leukemia, acute myeloid leukemia).

In embodiments, the cancer whose growth may be inhibited using a selective dipeptidyl peptidase inhibitor and an OX40 agonist are virally-associated cancers. Exemplary virally-associated cancers include, but are not limited to, cancers associated with Epstein-Barr virus (EBV), hepatitis B virus (HBV), hepatitis C virus (HCV), human papilloma viruses (HPV), merkel cell polyomavirus (MCV), human T lymphotropic virus type 1 (HTLV-1), human T lymphotropic type 2 (HTLV-2) and human herpesvirus, such as human herpesvirus 8 (HHV-8). The cancers associated with particular viruses are known to those of ordinary skill in the art. Examples of EBV-associated cancers include, but are not limited to, lymphomas, nasopharyngeal cancer, gastric carcinoma, parotid carcinoma, breast carcinoma, and leiomyosarcoma. Examples of cancers associated with hepatitis B virus (HBV) and hepatitis C virus (HCV) include but are not limited to cancers of the liver. Examples of cancers associated with human papilloma viruses (HPV) include, but are not limited to, oropharyngeal head and neck cancer, nasopharyngeal head and neck cancer, and cancers of the cervix, vulva, vagina, penis and anus. Examples of cancers associated with human T lymphotropic virus type 1 (HTLV-1) and type 2 (HTLV-2) include, but are not limited to, adult T-cell leukemia and hairy-cell leukemia, respectively. Examples of cancers associated with human herpesvirus 8 (HHV-8) include, but are not limited to, Kaposi sarcoma. Examples of cancers associated with merkel cell polyomavirus (MCV) include, but are not limited to, merkel cell carcinoma. In embodiments, the virally-associated cancer is a cancer associated with HPV. In other embodiments, the virally-associated cancer is a cancer associated with HCV.

In embodiments, the cancers whose growth may be inhibited using a selective dipeptidyl peptidase inhibitor, an OX40 agonist and a PD-1 axis inhibitor are virally-associated cancers. Exemplary virally-associated cancers include, but are not limited to, cancers associated with Epstein-Barr virus (EBV), hepatitis B virus (HBV), hepatitis C virus (HCV), human papilloma viruses (HPV), merkel cell polyomavirus (MCV), human T lymphotropic virus type 1 (HTLV-1), human T lymphotropic type 2 (HTLV-2) and human herpesvirus, such as human herpesvirus 8 (HHV-8). The cancers associated with particular viruses are known to those of ordinary skill in the art. Examples of EBV-associated cancers include, but are not limited to, lymphomas, nasopharyngeal cancer, gastric carcinoma, parotid carcinoma, breast carcinoma, and leiomyosarcoma. Examples of cancers associated with hepatitis B virus (HBV) and hepatitis C virus (HCV) include but are not limited to cancers of the liver. Examples of cancers associated with human papilloma viruses (HPV) include, but are not limited to, oropharyngeal head and neck cancer, nasopharyngeal head and neck cancer, and cancers of the cervix, vulva, vagina, penis and anus. Examples of cancers associated with human T lymphotropic virus type 1 (HTLV-1) and type 2 (HTLV-2) include, but are not limited to, adult T-cell leukemia and hairy-cell leukemia, respectively. Examples of cancers associated with human herpesvirus 8 (HHV-8) include, but are not limited to, Kaposi sarcoma. Examples of cancers associated with merkel cell polyomavirus (MCV) include, but are not limited to, merkel cell carcinoma. In embodiments, the virally-associated cancer is a cancer associated with HPV. In other embodiments, the virally-associated cancer is a cancer associated with HCV.

### Embodiments

Embodiment 1. A method of treating cancer in a subject in need thereof, the method comprising administering to the subject therapeutically effective amounts of a dipeptidyl peptidase inhibitor and an OX40 agonist.

Embodiment 2. A method of generating an anti-tumor immune response in a subject with cancer, the method comprising administering to the subject therapeutically effective amounts of a dipeptidyl peptidase inhibitor and an OX40 agonist.

Embodiment 3. The method of embodiment 1 or 2, further comprising administration of therapeutically effective amounts of one or more immune checkpoint inhibitors.

Embodiment 4. The method of any one of embodiments 1-3, wherein the dipeptidyl peptidase inhibitor is a compound or an antibody, preferably a compound.

Embodiment 5. The method of embodiment 4, wherein the compound is talabostat or an analog, a prodrug, a stereoisomer, or a pharmaceutically acceptable salt thereof.

Embodiment The method of embodiment 4, wherein the dipeptidyl peptidase inhibitor is talabostat or a pharmaceutically acceptable salt thereof.

Embodiment 7. The method of embodiment 4, wherein the dipeptidyl peptidase inhibitor is talabostat mesylate.

Embodiment 8. The method of embodiment 4, wherein the dipeptidyl peptidase inhibitor is an analog of talabostat.

Embodiment 9. The method of embodiment 4, wherein the dipeptidyl peptidase inhibitor is ARI-4175.

Embodiment 10. The method of embodiment 4, wherein the dipeptidyl peptidase inhibitor is a prodrug of talabostat.

Embodiment 11. The method of embodiment 4, wherein the dipeptidyl peptidase inhibitor is cyclohexyl(glycinyl)-prolinyl-valinyl-L-boroproline

Embodiment 12. The method of any one of embodiments 1-11, wherein the OX40 agonist is selected from the group consisting of an antibody, an oligomeric or multimeric molecule, a fusion protein, an OX40L agonist fragment and an immunoadhesin.

Embodiment 13. The method of embodiment 12, wherein the OX40 agonist is an antibody.

Embodiment 14. The method of any one of embodiments 1-11, wherein the OX40 agonist is selected from the group consisting of PF-04518600, pogalizumab (MOXR0916, RG 7888), MEDI6469, L106, ACT35, OX86, MEDI0562 (tavolixizumab, tavolimab), INCAGN01949 and GSK3174998.

Embodiment 15. The method of any one of embodiments 1-11, wherein the OX40 agonist is PF-04518600.

Embodiment 16. The method of any one of embodiments 3-15, wherein the one or more immune checkpoint inhibitors is a PD-1 axis inhibitor and/or a CTLA4 inhibitor.

Embodiment 17. The method of embodiment 16, wherein the PD-1 axis inhibitor comprises a PD-1 inhibitor, a PD-L1 inhibitor, or a PD-L2 inhibitor.

Embodiment 18. The method of any one of embodiments 3-15, wherein the one or more immune checkpoint inhibitors is a PD-1 inhibitor selected from the group consisting of ANA011, AUNP-12, tislelizumab (BGB-A317), KD033, pembrolizumab, MCLA-134, mDX400, MEDI0680, muDX400, nivolumab, spartalizumab (PDR001), sasanlimab (PF-06801591), cemiplimab (semiprimab, REGN-2810), camrelizumab (SHR 1210), STI-Al110, dostarlimab (TSR-042 or TSR042 or ANB0ll), 244C8, 388D4, prolgolimab (BCD100), camrelizumab (SHR 1210), cetrelimab (JNJ63723283), JS001, XCE853, GLS-010 (AB-122; WBP-3055), sintilimab (IBI-308), genolimzumab (CBT-501, GB226, APL-501), AK-103, theralizumab (TGN1412, CD28-SuperMAB, TAB-08 and TAB08), BI-754091, INCMGA00012 (MGA 012, INCMGA-0012), ABBV-181 (budigalimab), CC-90006 (C-90006), AGEN-2034w (AGEN-2034), LZM-009, Sym021, AK-105, CS1003, HLX-10 and AMP-224, preferably pembrolizumab or nivolumab.

Embodiment 19. The method of any one of embodiments 3-15, wherein the one or more immune checkpoint inhibitors is a PD-L1 inhibitor selected from a group consisting of avelumab, BMS-936559, BMS-986189, CA-170, CK-301 (cosibelimab), lodapolimab (LY-3300054), CX-072, CBT-502 (TQB2450), FAZ-053, FS118, HTI-1088 (HTI-1316; SHR 1316), MSB 2311, BGB-A333, IMC-001(STI-3031; STI-A1015KN035), HLX-20, A 167 (HBM-9167; KL-A167), KD033, durvalumab, KN035, MCLA-145, SP142, STI-A1011, STI-A1012, STI-A1010, STI-A1013, STI-A1014, STI-A1015, A110, KY1003, KD033 and atezolizumab, preferably avelumab.

Embodiment 20. The method of any one of embodiments 3-15, wherein the one or more immune checkpoint inhibitors is the PD-L2 inhibitor rHIgM12B7.

Embodiment 21. The method of any one of embodiments 3-15, wherein the one or more immune checkpoint inhibitors is a CTLA4 inhibitor selected from the group consisting of KAHR-102, AGEN1884, KN044, BMS-986218, MK-1308, ADU-1604, BMS-986249, CS-1002, BCD-145, REGN-4659, tremelimumab and ipilimumab, preferably tremelimumab or ipilimumab.

Embodiment 22. The method of any one of embodiments 1-21, wherein the dipeptidyl peptidase inhibitor is administered at a dose from about 0.001 mg/kg to about 1 mg/kg, preferably about 0.001 mg/kg to about 0.05 mg/kg, or more preferably about 0.001 mg/kg to about 0.035 mg/kg.

Embodiment 23. The method of any one of embodiments 1-22, wherein the OX40 agonist is administered at a dose from about 0.01 mg/kg to about 20 mg/kg body, preferably about 0.1 mg/kg to about 10 mg/kg, or more preferably about 0.1 mg/kg to about 5 mg/kg.

Embodiment 24. The method of embodiment 17 or 18, wherein the PD-1 inhibitor is administered at a dose of about 0.1 mg/kg to about 20 mg/kg, preferably about 0.3 mg/kg to about 10 mg/kg, or more preferably about 1 mg/kg to about 3 mg/kg.

Embodiment 25. The method of any one of embodiments 1-24, wherein the dipeptidyl peptidase inhibitor and the OX40 agonist are administered together as part of a single dosage form.

Embodiment 26. The method of any one of embodiments 1-24, wherein the dipeptidyl peptidase inhibitor and the OX40 agonist are administered together as two separate dosage forms.

Embodiment 27. The method of any one of embodiments 3-24, wherein the dipeptidyl peptidase inhibitor, the OX40 agonist and one or more immune checkpoint inhibitors are administered together as part of a single dosage form.

Embodiment 28. The method of any one of embodiments 3-24, wherein the dipeptidyl peptidase inhibitor, the OX40 agonist and one or more immune checkpoint inhibitors are administered as three or more separate dosage forms.

Embodiment 29. The method of any one of embodiments 1-28, wherein the cancer is selected from the group consisting of melanoma, metastatic melanoma, oral squamous cell carcinoma, small cell lung cancer, breast cancer, colorectal cancer, colon cancer, pancreatic cancer, lung cancer, glioblastoma, hepatocellular carcinoma, head and neck cancer, leukemia, lymphoma, sarcoma, fibrosarcoma, lymphocytic leukemia, non-Hodgkin's lymphoma, Hodgkin's lymphoma, anaplastic large-cell lymphoma, myeloid leukemia, multiple myeloma, acute lymphoblastic leukemia, chronic myeloid leukemia, chronic lymphocytic leukemia, prostate cancer, neuroendocrine prostate cancer, hormone refractory prostate cancer, castration resistant prostate cancer, androgen resistant prostate cancer, treatment resistant prostate cancer and acute myeloid leukemia, preferably prostate cancer, pancreatic cancer and colorectal cancer.

Embodiment 30. The method of any one of embodiments 1-28, wherein the cancer is colorectal cancer.

Embodiment 31. The method of any one of embodiments 1-28, wherein the cancer is pancreatic cancer.

Embodiment 32. The method of any one of embodiments 1-28, wherein the cancer is prostate cancer.

Embodiment 33. The method of embodiment 1 or 2, wherein the dipeptidyl peptidase inhibitor is talabostat mesylate and the OX40 agonist is PF-04518600.

Embodiment 34. The method of embodiment 3, wherein the dipeptidyl peptidase inhibitor is talabostat mesylate and the OX40 agonist is PF-04518600.

Embodiment The method of embodiment 3, wherein the dipeptidyl peptidase inhibitor is talabostat mesylate, the OX40 agonist is PF-04518600, and the one or more immune checkpoint inhibitors is a PD-1 inhibitor and/or a CTLA4 inhibitor.

Embodiment 36. The method of embodiment 3, wherein the dipeptidyl peptidase inhibitor is talabostat mesylate, the OX40 agonist is PF-04518600, and the one or more immune checkpoint inhibitors is a PD-1 inhibitor.

Embodiment 37. The method of embodiment 3, wherein the dipeptidyl peptidase inhibitor is talabostat mesylate, the OX40 agonist is PF-04518600, and the immune checkpoint inhibitor is nivolumab.

Embodiment 38. The method of embodiment 3, wherein the dipeptidyl peptidase inhibitor is talabostat mesylate, the OX40 agonist is PF-04518600, and the immune checkpoint inhibitor is pembrolizumab.

Embodiment 39. The method of embodiment 3, wherein the dipeptidyl peptidase inhibitor is talabostat mesylate, the OX40 agonist is PF-04518600, and the immune checkpoint inhibitor is avelumab.

Embodiment 40. A pharmaceutical composition for the treatment of cancer comprising:
(i) a therapeutically effective amount of a dipeptidyl peptidase inhibitor,
(ii) a therapeutically effective amount of an OX40 agonist, and
(iii) one or more pharmaceutically acceptable carriers and/or excipients.

Embodiment 41. A pharmaceutical composition for the treatment of cancer comprising:
(i) a therapeutically effective amount of a dipeptidyl peptidase inhibitor,
(ii) a therapeutically effective amount of an OX40 agonist,
(iii) a therapeutically effective amount of one or more immune checkpoint inhibitors, and
(iv) one or more pharmaceutically acceptable carriers and/or excipients.

Embodiment 42. The pharmaceutical composition of embodiment 41, wherein the one or more immune checkpoint inhibitors comprises a PD-1 axis inhibitor and/or a CTLA4 inhibitor.

Embodiment 43. The pharmaceutical composition of embodiment 42, wherein the PD-1 axis inhibitor comprises a PD-1 inhibitor, a PD-L1 inhibitor, and/or a PD-L2 inhibitor.

Embodiment 44. The pharmaceutical composition of embodiment 41, wherein the immune checkpoint inhibitor is a PD-1 inhibitor selected from the group consisting of ANA011, AUNP-12, tislelizumab (BGB-A317), KD033, pembrolizumab, MCLA-134, mDX400, MEDI0680, muDX400, nivolumab, spartalizumab (PDR001), sasanlimab (PF-06801591), cemiplimab (Semiprimab, REGN-2810), camrelizumab (SHR 1210), STI-Al110, dostarlimab (TSR-042 or TSR042 or ANB0ll), 244C8, 388D4, prolgolimab (BCD100), cetrelimab (JNJ63723283), JS001, XCE853, GLS-010 (AB-122; WBP-3055), sintilimab (IBI-308), genolimzumab (CBT-501, GB226, APL-501), AK-103, theralizumab (TGN1412, CD28-SuperMAB, TAB-08 and TAB08), BI-754091, INCMGA00012 (MGA 012, INCMGA-0012), ABBV-181 (Budigalimab), CC-90006 (C-90006), AGEN-2034w (AGEN-2034), LZM-009, Sym021, AK-105, CS1003, HLX-10, and AMP-224, preferably pembrolizumab or nivolumab.

Embodiment 45. The pharmaceutical composition of embodiment 41, wherein the immune checkpoint inhibitor is a PD-L1 inhibitor selected from the group consisting of avelumab, BMS-936559, BMS-986189, CA-170, durvalumab, KN035, MCLA-145, SP142, STI-A1011, STI-A1012, STI-A1010, STI-A1013, STI-A1014, STI-A1015, A110, KY1003, KD033 and atezolizumab, preferably avelumab.

Embodiment 46. The pharmaceutical composition of embodiment 41, wherein the immune checkpoint inhibitor is the PD-L2 inhibitor rHIgM12B7.

Embodiment 47. The pharmaceutical composition of embodiment 41, wherein the immune checkpoint inhibitor is a CTLA-4 inhibitor selected from the group consisting of KAHR-102, AGEN1884, BMS-986218, MK-1308, ADU-1604, BMS-986249, CS-1002, BCD-145, REGN-4659, KN044, tremelimumab and ipilimumab, preferably tremelimumab or ipilimumab.

Embodiment 48. The pharmaceutical composition of any one of embodiments 40-47, wherein the dipeptidyl peptidase inhibitor is talabostat mesylate.

Embodiment 49. The pharmaceutical composition of any one of embodiments 40-48, wherein the OX40 agonist is selected from the group consisting of PF-04518600, pogalizumab (MOXR0916, RG7888), MEDI6469, efizonerimod alfa (MEDI 6383), L106 BD, ACT35, OX86, MEDI0562 (tavolixizumab/tavolimab), INCAGN01949, and GSK3174998, preferably PF-04518600.

Embodiment 50. The pharmaceutical composition of any one of embodiments 40-49, wherein the pharmaceutical composition is administered together as part of a single dosage form.

Embodiment 51. The pharmaceutical composition of any one of embodiments 40-49, wherein the pharmaceutical composition is administered together as two or more separate dosage forms.

Embodiment 52. The pharmaceutical composition of any one of embodiments 40-51, wherein the pharmaceutical composition is administered by the oral or parenteral route.

Embodiment 53. A kit comprising:
(i) a single dose or multiple doses of a dipeptidyl peptidase inhibitor,
(ii) a single dose or multiple doses of an OX40 agonist, and
(iii) instructions for using the dipeptidyl peptidase inhibitor and OX40 agonist to treat a subject with cancer.

Embodiment 54. A kit comprising:
(i) a single dose or multiple doses of a dipeptidyl peptidase inhibitor,
(ii) a single dose or multiple doses of an OX40 agonist,
(iii) a single dose or multiple doses of one or more immune check point inhibitors, and
(iv) instructions for using the dipeptidyl peptidase inhibitor, OX40 agonist and immune checkpoint inhibitor(s) to treat a subject with cancer.

Embodiment 55. The kit according to embodiment 54, wherein the immune check point inhibitor is a PD-1 axis inhibitor or a CTLA4 inhibitor.

Embodiment 56. The kit according to embodiment 55, wherein the PD-1 axis inhibitor is a PD-1 inhibitor.

Embodiment 57. The kit according to embodiment 54, wherein the immune check point inhibitor is a PD-1 inhibitor selected from the group consisting of ANA011, AUNP-12, tislelizumab (BGB-A317), KD033, pembrolizumab, MCLA-134, mDX400, MEDI0680, muDX400, nivolumab, spartalizumab (PDR001), sasanlimab (PF-06801591), cemiplimab (Semiprimab, REGN-2810), camrelizumab (SHR 1210), STI-Al110, dostarlimab (TSR-042 or TSR042 or ANB0ll), 244C8, 388D4, prolgolimab (BCD100), cetrelimab (JNJ63723283), JS001 XCE853, GLS-010 (AB-122; WBP-3055), sintilimab (IBI-308), genolimzumab (CBT-501, GB226, APL-501), AK-103, theralizumab (TGN1412, CD28-SuperMAB, TAB-08 and TAB08), BI-754091, INCMGA00012 (MGA 012, INCMGA-0012), ABBV-181 (Budigalimab), CC-90006 (C-90006), AGEN-2034w (AGEN-2034), LZM-009, Sym021, AK-105, CS1003, HLX-10, and AMP-224, preferably pembrolizumab or nivolumab.

Embodiment 58. The kit according to embodiment 54, wherein the immune check point inhibitor is a CTLA4 inhibitor selected from the group consisting of KAHR-102, AGEN1884, BMS-986218, MK-1308, ADU-1604, BMS-986249, CS-1002, BCD-145, REGN-4659, KN044, tremelimumab and ipilimumab, preferably tremelimumab or ipilimumab.

Embodiment 59. The kit according to any one of embodiments 53-58, wherein the dipeptidyl peptidase inhibitor is talabostat mesylate.

Embodiment 60. The kit according to any one of embodiments 53-59, wherein the OX40 agonist is PF-04518600.

### EXAMPLES

### Example 1: Talabostat Mesylate and OX40 Agonist Antibody with or without Anti-PD-1 Antibody Induces a Significant Anti-Tumor Response in an MC38 Mouse Model of Adenocarcinoma

### Materials and Methods

Animals: Six to ten week-old female C57BL/6 mice were used in the studies as supplied by Beijing Vital River Laboratory Animal Technology Co., Ltd. Mice received food and water ad libitum. All animals were maintained in a controlled environment with 20-26° C. temperature, 40-70% humidity, and a light/dark cycle of 12 hours each. Up to 5 mice were kept in each cage. The study protocol and procedures involving the care and use of animals were reviewed and approved by the Institutional Animal Care and Use Committee (IACUC) to ensure compliance with the regulations of the Association for Assessment and Accreditation of Laboratory Animal Care (AAALAC).

Reagents and Antibodies: Anti-mouse PD-1 antibody (BioXcell; Lot No./Cat. No./clone: 665418F1/BP0146/RMP1-14) was supplied at a concentration of 7.83 mg/mL and maintained at 4° C. Dosing solutions of anti-mouse PD-1 antibody were freshly prepared at a concentration of 0.5 mg/mL in sterile phosphate buffered saline (PBS), pH 7.0 and administered at a dose of 5 mg/kg, intraperitoneally (i.p) per mouse. Talabostat mesylate (Aptuit, Ltd.) was was prepared at a stock concentration of 31 mg/mL in hydrochloric acid and maintained at −20° C. Fresh dosing solutions of talabostat mesylate were prepared at a working concentration of 0.1 mg/mL before every administration in normal saline and administered perorally (p.o) at a dose of 20 μg per mouse. Anti-mouse OX40 agonist antibody (BioXCell; Lot No./Cat. No./clone: 672418M2/BP0031/OX-86) was supplied at a concentration of 8.46 mg/mL and maintained at 4° C. Dosing solutions of anti-mouse OX-40 antibody were freshly prepared at a working concentration of 1 mg/mL in PBS, and administered at a dose of 10 mg/kg intraperitoneally per mouse.

Tumor Model: MC38 cells were inoculated into C57BL/6 mice. The date of tumor cell inoculation is denoted as Day 0. Mean tumor volume was about 129 mm³ when treatment was initiated at 10 days post-implant. At the ten day time point, mice were sorted into groups of 10 and were administered talabostat mesylate, anti-PD-1 and/or anti-OX40 antibody according to the dosing described in Table 4 below. The mice received treatment for 28 days (until day 38 of the study).

TABLE 4
Treatment groups, dosing route and schedule

| | | | | Dosing | |
|---|---|---|---|---|---|
| Group | N | Treatment | Dose | Route | Dosing Schedule |
| 1 | 10 | Talabostat | NA | p.o | QD |
| | | mesylate | | | (Day 11 to Day 38) |
| | | vehicle | | | |
| | | control | | | |
| | | Vehicle | NA | i.p | Day 11, Day 14, Day |
| | | control | | | 18, Day 21, Day 25, |
| | | | | | Day 28, Day 32, Day |
| | | | | | 35 |
| 2 | 10 | Talabostat | 20 μg/ | p.o. | QD |
| | | mesylate | mouse | | (Day 11 to Day 38) |
| 3 | 10 | Anti-OX40 | 10 mg/kg | i.p. | Day 11, Day 14, Day |
| | | Ab | | | 18, Day 21, Day 25, |
| | | | | | Day 28, Day 32, Day |
| | | | | | 35 |
| 4 | 10 | Talabostat | 20 μg/ | p.o. | QD |
| | | mesylate | mouse | | (Day 11 to Day 38) |
| | | Anti-PD-1 | 5 mg/kg | i.p. | Day 11, Day 14, Day |
| | | Ab | | | 18, Day 21, Day 25, |
| | | | | | Day 28, Day 32, Day |
| | | | | | 35 |
| 5 | 10 | Anti-PD-1 | 5 mg/kg | i.p. | Day 11, Day 14, Day |
| | | Ab | | | 18, Day 21, Day 25, |
| | | | | | Day 28, Day 32, Day |
| | | | | | 35 |
| | | Anti-OX40 | 10 mg/kg | i.p. | Day 11, Day 14, Day |
| | | Ab | | | 18, Day 21, Day 25, |
| | | | | | Day 28, Day 32, Day |
| | | | | | 35 |
| 6 | 10 | Anti-OX40 | 10 mg/kg | i.p. | Day 11, Day 14, Day |
| | | Ab | | | 18, Day 21, Day 25, |
| | | | | | Day 28, Day 32, Day |
| | | | | | 35 |
| | | Talabostat | 20 μg/ | p.o. | QD |
| | | mesylate | mouse | | (Day 11 to Day 38) |
| 7 | 10 | Anti-OX40 | 10 mg/kg | i.p. | Day 11, Day 14, Day |
| | | Ab | | | 18, Day 21, Day 25, |
| | | | | | Day 28, Day 32, Day |
| | | | | | 35 |
| | | Talabostat | 20 μg/ | p.o. | QD |
| | | mesylate | mouse | | (Day 11 to Day 38) |
| | | Anti-PD-1 | 5 mg/kg | i.p. | Day 11, Day 14, Day |
| | | Ab | | | 18, Day 21, Day 25, |
| | | | | | Day 28, Day 32, Day |
| | | | | | 35 |

N: number of mice, QD: once daily, Ab: Antibody.

Tumor volumes were measured on Day 10, Day 14, Day 17, Day 21, Day 23, Day 25, Day 28, Day 30, Day 32, Day 35, Day 37, Day 39, Day 42, Day 44, Day 46, Day 49 and Day 51 in two dimensions using a caliper, and the volume was expressed in mm³ using the formula: V=0.5 a×b² where a and b are the length and width of the tumor, respectively.

Statistical Analysis: Data related to tumor volume are presented as mean and the standard error of the mean (SEM). Statistical analyses were conducted using Student's t-test. P<0.05 was considered statistically significant. Percentage tumor reduction was assessed on Day 23 by using the below formula:

% Tumor reduction=(Mean tumor volume^{vehicle control}−Mean tumor volume^{treatment group})/Mean tumor volume^{vehicle control}×100

### Results

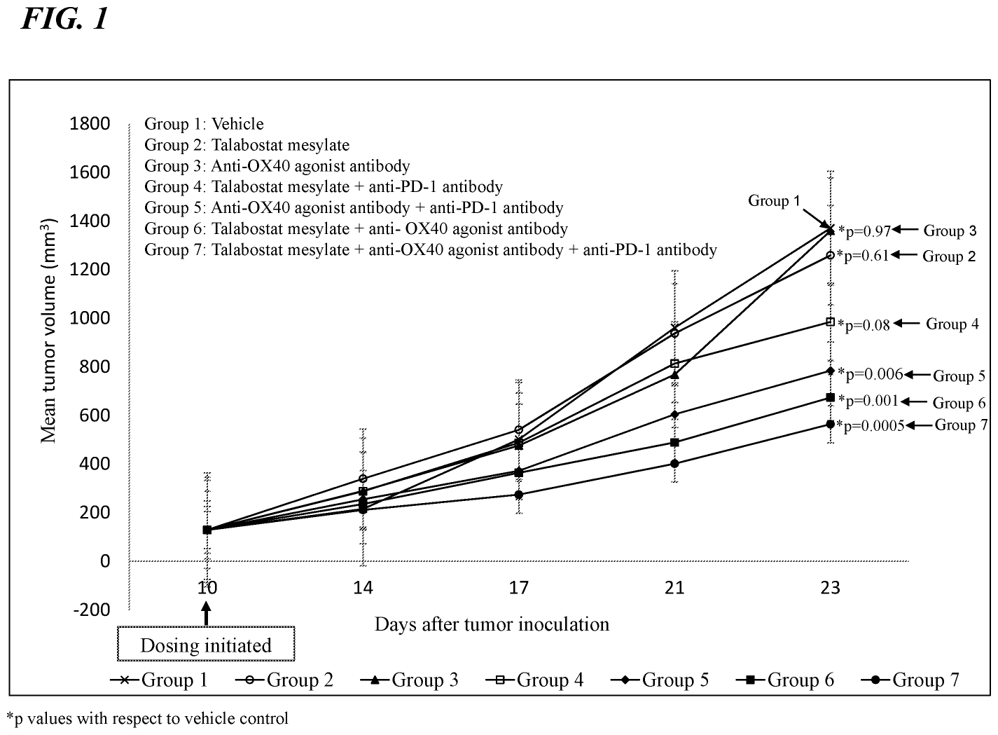

Tumor burden: Tumor-bearing mice treated with talabostat mesylate (20 μg per mouse, qd) and anti-OX40 agonist antibody (10 mg/kg, twice weekly) exhibited a significant reduction in tumor burden on day 23 compared to either talabostat mesylate, anti-OX40 agonist antibody alone (group 6 vs. group 2, p=0.001 and group 6 vs. group 3, p=0.005) or the vehicle control (group 6 vs. group 1, p=0.0010). Further, mice treated with talabostat mesylate (20 μg per mouse, qd), anti-OX40 agonist antibody (10 mg/kg, twice weekly) and anti-PD-1 antibody (5 mg/kg, twice weekly) showed a significant decrease in tumor burden on day 23 as compared to talabostat mesylate and anti-PD-1 antibody (group 7 vs. group 4, p=0.01), talabostat mesylate alone (group 7 vs. group 2, p=0.0005), OX-40 agonist antibody alone (group 7 vs. group 3, p=0.002) and the vehicle control (group 7 vs. group 1, p=0.0005). The results are summarized in FIG. 1 and Table 5 below.

TABLE 5
Combination therapy reduced tumor burden in MC38 mouse model

| | % Tumor Reduction | |
|---|---|---|
| | compared to vehicle | |
| Groups | control on Day 23 | |
| Talabostat mesylate, Group 2 | 8.16 | (p = 0.61) |
| Anti-OX40 agonist antibody, Group 3 | 0.80 | (p = 0.97) |
| Talabostat mesylate + anti-PD-1 antibody, Group 4 | 28.15 | (p = 0.08) |
| OX40 agonist antibody + anti-PD-1 antibody, Group | 42.79 | (p = 0.006) |
| 5 | | |
| Talabostat mesylate + anti-OX40 agonist antibody, | 50.85 | (p = 0.0010) |
| Group 6 | | |
| Talabostat mesylate + anti-OX40 agonist antibody + | 58.84 | (p = 0.0005) |
| anti-PD-1 antibody, Group 7 | | |

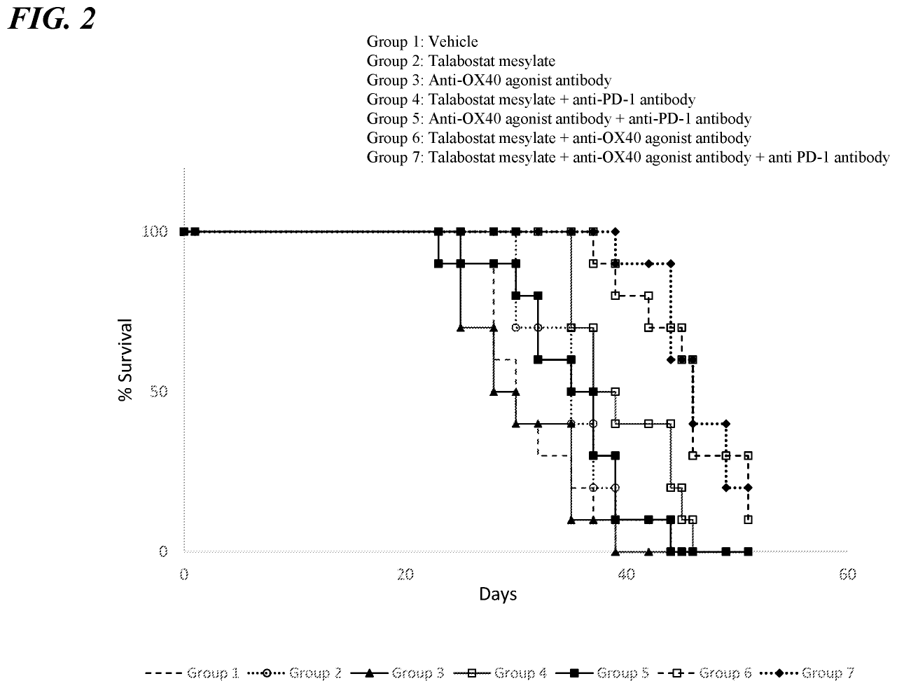

Survival: Furthermore, tumor-bearing mice treated with talabostat mesylate and anti-OX40 agonist antibody demonstrated a significant improvement in survival. The median survival of the vehicle control (group 1) treated animals was 30 days. Talabostat mesylate (group 2) or anti-OX40 agonist antibody (group 3) treated animals showed a median survival of 35 days and 28 days, respectively (group 2 vs. group 1, p=0.0958; group 3 vs. group 1, p=0.8560). The median survival of animals treated with talabostat mesylate and anti-OX40 agonist antibody (group 6) was significantly increased to 46 days in comparison to talabostat mesylate alone (group 6 vs. group 2, p<0.001), while combination of anti-PD-1 antibody and OX40 agonist antibody-treated animals (group 5) had a median survival of 35 days (group 5 vs. group 1, p=0.0991). The triple combination of talabostat mesylate, OX40 agonist antibody and anti-PD-1 antibody (group 7) resulted in a median survival of 46 days (group 7 vs group 1, p<0.001). The results are shown in FIG. 2.

Various cytokines and chemokines in the serum of animals treated with talabostat mesylate alone was also evaluated. Talabostat mesylate stimulated several pro-inflammatory cytokines and chemokines, including IL-18 (data not shown). IL-18 bridges the innate and adaptive immune systems through induction of IFNγ and OX40 (CD134) signaling pathway (Maxwell et al., Journal of Immunology, 2006, 177:234). Thus, it is postulated that talabostat mesylate regulates cytokine pathways that synergize with OX40 agonist immunotherapy for the treatment of solid cancer.

In conclusion, the presence of talabostat mesylate in the drug combinations was necessary for a survival benefit. While the combination of anti-OX40 agonist and anti-PD-1 antibodies showed a significant reduction in tumor volume on day 23 (Table 5 and FIG. 1), this did not translate into an overall survival benefit (FIG. 2). Talabostat mesylate in combination with anti-OX40 agonist and anti-PD-1 antibodies contributed to a clear synergistic anti-tumor response that dramatically increased survival. The present invention supports the clinical evaluation of talabostat mesylate in combination with an anti-OX40 agonist antibody with or without an anti-PD-1 antibody for the treatment of cancer.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. A method of treating cancer in a subject in need thereof, the method comprising administering to the subject therapeutically effective amounts of a dipeptidyl peptidase inhibitor and an OX40 agonist.

2. A method of generating an anti-tumor immune response in a subject with cancer, the method comprising administering to the subject therapeutically effective amounts of a dipeptidyl peptidase inhibitor and an OX40 agonist.

3. The method of claim 1 or 2, further comprising administration of therapeutically effective amounts of one or more immune checkpoint inhibitors.

4. The method of any one of claims 1-3, wherein the dipeptidyl peptidase inhibitor is selected from the group consisting of a compound or an antibody, preferably a compound.

5. The method of claim 4, wherein the compound is talabostat or an analog, a prodrug, a stereoisomer, or a pharmaceutically acceptable salt thereof.

6. The method of claim 4, wherein the dipeptidyl peptidase inhibitor is talabostat or a pharmaceutically acceptable salt thereof.

7. The method of claim 4, wherein the dipeptidyl peptidase inhibitor is talabostat mesylate.

8. The method of claim 4, wherein the dipeptidyl peptidase inhibitor is an analog of talabostat.

9. The method of claim 4, wherein the dipeptidyl peptidase inhibitor is ARI-4175.

10. The method of claim 4, wherein the dipeptidyl peptidase inhibitor is a prodrug of talabostat.

11. The method of claim 4, wherein the dipeptidyl peptidase inhibitor is cyclohexyl(glycinyl)-prolinyl-valinyl-L-boroproline

12. The method of any one of claims 1-11, wherein the OX40 agonist is selected from the group consisting of an antibody, an oligomeric or multimeric molecule, a fusion protein, an OX40L agonist fragment and an immunoadhesin.

13. The method of claim 12, wherein the OX40 agonist is an antibody.

14. The method of any one of claims 1-11, wherein the OX40 agonist is selected from the group consisting of PF-04518600, pogalizumab (MOXR0916, RG 7888), MEDI6469, L106, ACT35, OX86, MEDI0562 (tavolixizumab, tavolimab), INCAGN01949 and GSK3174998.

15. The method of any one of claims 1-11, wherein the OX40 agonist is PF-04518600.

16. The method of any one of claims 3-15, wherein the one or more immune checkpoint inhibitors is a PD-1 axis inhibitor and/or a CTLA4 inhibitor.

17. The method of claim 16, wherein the PD-1 axis inhibitor comprises a PD-1 inhibitor, a PD-L1 inhibitor, or a PD-L2 inhibitor.

18. The method of any one of claims 3-15, wherein the one or more immune checkpoint inhibitors is a PD-1 inhibitor selected from the group consisting of ANA011, AUNP-12, tislelizumab (BGB-A317), KD033, pembrolizumab, MCLA-134, mDX400, MEDI0680, muDX400, nivolumab, spartalizumab (PDR001), sasanlimab (PF-06801591), cemiplimab (semiprimab, REGN-2810), camrelizumab (SHR 1210), STI-Al110, dostarlimab (TSR-042 or TSR042 or ANB0ll), 244C8, 388D4, prolgolimab (BCD100), camrelizumab (SHR 1210), cetrelimab (JNJ63723283), JS001, XCE853, GLS-010 (AB-122; WBP-3055), sintilimab (IBI-308), genolimzumab (CBT-501, GB226, APL-501), AK-103, theralizumab (TGN1412, CD28-SuperMAB, TAB-08 and TAB08), BI-754091, INCMGA00012 (MGA 012, INCMGA-0012), ABBV-181 (budigalimab), CC-90006 (C-90006), AGEN-2034w (AGEN-2034), LZM-009, Sym021, AK-105, CS1003, HLX-10 and AMP-224, preferably pembrolizumab or nivolumab.

19. The method of any one of claims 3-15, wherein the one or more immune checkpoint inhibitors is a PD-L1 inhibitor selected from a group consisting of avelumab, BMS-936559, BMS-986189, CA-170, CK-301 (cosibelimab), lodapolimab (LY-3300054), CX-072, CBT-502 (TQB2450), FAZ-053, FS118, HTI-1088 (HTI-1316; SHR 1316), MSB 2311, BGB-A333, IMC-001(STI-3031; STI-A1015KN035), HLX-20, A 167 (HBM-9167; KL-A167), KD033, durvalumab, KN035, MCLA-145, SP142, STI-A1011, STI-A1012, STI-A1010, STI-A1013, STI-A1014, STI-A1015, A110, KY1003, KD033 and atezolizumab, preferably avelumab.

20. The method of any one of claims 3-15, wherein the one or more immune checkpoint inhibitors is the PD-L2 inhibitor rHIgM12B7.

21. The method of any one of claims 3-15, wherein the one or more immune checkpoint inhibitors is a CTLA4 inhibitor selected from the group consisting of KAHR-102, AGEN1884, KN044, BMS-986218, MK-1308, ADU-1604, BMS-986249, CS-1002, BCD-145, REGN-4659, tremelimumab and ipilimumab, preferably tremelimumab or ipilimumab.

22. The method of any one of claims 1-21, wherein the dipeptidyl peptidase inhibitor is administered at a dose from about 0.001 mg/kg to about 1 mg/kg, preferably about 0.001 mg/kg to about 0.05 mg/kg, or more preferably about 0.001 mg/kg to about 0.035 mg/kg.

23. The method of any one of claims 1-22, wherein the OX40 agonist is administered at a dose from about 0.01 mg/kg to about 20 mg/kg body, preferably about 0.1 mg/kg to about 10 mg/kg, or more preferably about 0.1 mg/kg to about 5 mg/kg.

24. The method of claim 17 or 18, wherein the PD-1 inhibitor is administered at a dose of about 0.1 mg/kg to about 20 mg/kg, preferably about 0.3 mg/kg to about 10 mg/kg, or more preferably about 1 mg/kg to about 3 mg/kg.

25. The method of any one of claims 1-24, wherein the dipeptidyl peptidase inhibitor and the OX40 agonist are administered together as part of a single dosage form.

26. The method of any one of claims 1-24, wherein the dipeptidyl peptidase inhibitor and the OX40 agonist are administered together as two separate dosage forms.

27. The method of any one of claims 3-24, wherein the dipeptidyl peptidase inhibitor, the OX40 agonist and one or more immune checkpoint inhibitors are administered together as part of a single dosage form.

28. The method of any one of claims 3-24, wherein the dipeptidyl peptidase inhibitor, the OX40 agonist and one or more immune checkpoint inhibitors are administered as three or more separate dosage forms.

29. The method of any one of claims 1-28, wherein the cancer is selected from the group consisting of melanoma, metastatic melanoma, oral squamous cell carcinoma, small cell lung cancer, breast cancer, colorectal cancer, colon cancer, pancreatic cancer, lung cancer, glioblastoma, hepatocellular carcinoma, head and neck cancer, leukemia, lymphoma, sarcoma, fibrosarcoma, lymphocytic leukemia, non-Hodgkin's lymphoma, Hodgkin's lymphoma, anaplastic large-cell lymphoma, myeloid leukemia, multiple myeloma, acute lymphoblastic leukemia, chronic myeloid leukemia, chronic lymphocytic leukemia, prostate cancer, neuroendocrine prostate cancer, hormone refractory prostate cancer, castration resistant prostate cancer, androgen resistant prostate cancer, treatment resistant prostate cancer and acute myeloid leukemia, preferably prostate cancer, pancreatic cancer and colorectal cancer.

30. The method of any one of claims 1-28, wherein the cancer is colorectal cancer.

31. The method of any one of claims 1-28, wherein the cancer is pancreatic cancer.

32. The method of any one of claims 1-28, wherein the cancer is prostate cancer.

33. The method of claim 1 or 2, wherein the dipeptidyl peptidase inhibitor is talabostat mesylate and the OX40 agonist is PF-04518600.

34. The method of claim 3, wherein the dipeptidyl peptidase inhibitor is talabostat mesylate and the OX40 agonist is PF-04518600.

35. The method of claim 3, wherein the dipeptidyl peptidase inhibitor is talabostat mesylate, the OX40 agonist is PF-04518600, and the one or more immune checkpoint inhibitors is a PD-1 inhibitor and/or a CTLA4 inhibitor.

36. The method of claim 3, wherein the dipeptidyl peptidase inhibitor is talabostat mesylate, the OX40 agonist is PF-04518600, and the one or more immune checkpoint inhibitors is a PD-1 inhibitor.

37. The method of claim 3, wherein the dipeptidyl peptidase inhibitor is talabostat mesylate, the OX40 agonist is PF-04518600, and the immune checkpoint inhibitor is nivolumab.

38. The method of claim 3, wherein the dipeptidyl peptidase inhibitor is talabostat mesylate, the OX40 agonist is PF-04518600, and the immune checkpoint inhibitor is pembrolizumab.

39. The method of claim 3, wherein the dipeptidyl peptidase inhibitor is talabostat mesylate, the OX40 agonist is PF-04518600, and the immune checkpoint inhibitor is avelumab.

40. A pharmaceutical composition for the treatment of cancer comprising:
(i) a therapeutically effective amount of a dipeptidyl peptidase inhibitor,
(ii) a therapeutically effective amount of an OX40 agonist, and
(iii) one or more pharmaceutically acceptable carriers and/or excipients.

41. A pharmaceutical composition for the treatment of cancer comprising:
(i) a therapeutically effective amount of a dipeptidyl peptidase inhibitor,
(ii) a therapeutically effective amount of an OX40 agonist,
(iii) a therapeutically effective amount of one or more immune checkpoint inhibitors, and
(iv) one or more pharmaceutically acceptable carriers and/or excipients.

42. The pharmaceutical composition of claim 41, wherein the one or more immune checkpoint inhibitors comprises a PD-1 axis inhibitor and/or a CTLA4 inhibitor.

43. The pharmaceutical composition of claim 42, wherein the PD-1 axis inhibitor comprises a PD-1 inhibitor, a PD-L1 inhibitor, and/or a PD-L2 inhibitor.

44. The pharmaceutical composition of claim 41, wherein the immune checkpoint inhibitor is a PD-1 inhibitor selected from the group consisting of ANA011, AUNP-12, tislelizumab (BGB-A317), KD033, pembrolizumab, MCLA-134, mDX400, MEDI0680, muDX400, nivolumab, spartalizumab (PDR001), sasanlimab (PF-06801591), cemiplimab (Semiprimab, REGN-2810), camrelizumab (SHR 1210), STI-Al110, dostarlimab (TSR-042 or TSR042 or ANB0ll), 244C8, 388D4, prolgolimab (BCD100), cetrelimab (JNJ63723283), JS001, XCE853, GLS-010 (AB-122; WBP-3055), sintilimab (IBI-308), genolimzumab (CBT-501, GB226, APL-501), AK-103, theralizumab (TGN1412, CD28-SuperMAB, TAB-08 and TAB08), BI-754091, INCMGA00012 (MGA 012, INCMGA-0012), ABBV-181 (Budigalimab), CC-90006 (C-90006), AGEN-2034w (AGEN-2034), LZM-009, Sym021, AK-105, CS1003, HLX-10, and AMP-224, preferably pembrolizumab or nivolumab.

45. The pharmaceutical composition of claim 41, wherein the immune checkpoint inhibitor is a PD-L1 inhibitor selected from the group consisting of avelumab, BMS-936559, BMS-986189, CA-170, durvalumab, KN035, MCLA-145, SP142, STI-A1011, STI-A1012, STI-A1010, STI-A1013, STI-A1014, STI-A1015, A110, KY1003, KD033 and atezolizumab, preferably avelumab.

46. The pharmaceutical composition of claim 41, wherein the immune checkpoint inhibitor is the PD-L2 inhibitor rHIgM12B7.

47. The pharmaceutical composition of claim 41, wherein the immune checkpoint inhibitor is a CTLA-4 inhibitor selected from the group consisting of KAHR-102, AGEN1884, BMS-986218, MK-1308, ADU-1604, BMS-986249, CS-1002, BCD-145, REGN-4659, KN044, tremelimumab and ipilimumab, preferably tremelimumab or ipilimumab.

48. The pharmaceutical composition of any one of claims 40-47, wherein the dipeptidyl peptidase inhibitor is talabostat mesylate.

49. The pharmaceutical composition of any one of claims 40-48, wherein the OX40 agonist is selected from the group consisting of PF-04518600, pogalizumab (MOXR0916, RG7888), MEDI6469, efizonerimod alfa (MEDI 6383), L106 BD, ACT35, OX86, MEDI0562 (tavolixizumab/tavolimab), INCAGN01949, and GSK3174998, preferably PF-04518600.

50. The pharmaceutical composition of any one of claims 40-49, wherein the pharmaceutical composition is administered together as part of a single dosage form.

51. The pharmaceutical composition of any one of claims 40-49, wherein the pharmaceutical composition is administered together as two or more separate dosage forms.

52. The pharmaceutical composition of any one of claims 40-51, wherein the pharmaceutical composition is administered by the oral or parenteral route.

53. A kit comprising:
(i) a single dose or multiple doses of a dipeptidyl peptidase inhibitor,
(ii) a single dose or multiple doses of an OX40 agonist, and
(iii) instructions for using the dipeptidyl peptidase inhibitor and OX40 agonist to treat a subject with cancer.

54. A kit comprising:
(i) a single dose or multiple doses of a dipeptidyl peptidase inhibitor,
(ii) a single dose or multiple doses of an OX40 agonist,
(iii) a single dose or multiple doses of one or more immune check point inhibitors, and
(iv) instructions for using the dipeptidyl peptidase inhibitor, OX40 agonist and immune checkpoint inhibitor(s) to treat a subject with cancer.

55. The kit according to claim 54, wherein the immune check point inhibitor is a PD-1 axis inhibitor or a CTLA4 inhibitor.

56. The kit according to claim 55, wherein the PD-1 axis inhibitor is a PD-1 inhibitor.

57. The kit according to claim 54, wherein the immune check point inhibitor is a PD-1 inhibitor selected from the group consisting of ANA011, AUNP-12, tislelizumab (BGB-A317), KD033, pembrolizumab, MCLA-134, mDX400, MEDI0680, muDX400, nivolumab, spartalizumab (PDR001), sasanlimab (PF-06801591), cemiplimab (Semiprimab, REGN-2810), camrelizumab (SHR 1210), STI-Al110, dostarlimab (TSR-042 or TSR042 or ANB0ll), 244C8, 388D4, prolgolimab (BCD100), cetrelimab (JNJ63723283), JS001 XCE853, GLS-010 (AB-122; WBP-3055), sintilimab (IBI-308), genolimzumab (CBT-501, GB226, APL-501), AK-103, theralizumab (TGN1412, CD28-SuperMAB, TAB-08 and TAB08), BI-754091, INCMGA00012 (MGA 012, INCMGA-0012), ABBV-181 (Budigalimab), CC-90006 (C-90006), AGEN-2034w (AGEN-2034), LZM-009, Sym021, AK-105, CS1003, HLX-10, and AMP-224, preferably pembrolizumab or nivolumab.

58. The kit according to claim 54, wherein the immune check point inhibitor is a CTLA4 inhibitor selected from the group consisting of KAHR-102, AGEN1884, BMS-986218, MK-1308, ADU-1604, BMS-986249, CS-1002, BCD-145, REGN-4659, KN044, tremelimumab and ipilimumab, preferably tremelimumab or ipilimumab.

59. The kit according to any one of claims 53-58, wherein the dipeptidyl peptidase inhibitor is talabostat mesylate.

60. The kit according to any one of claims 53-59, wherein the OX40 agonist is PF-04518600.
